SEQUENCE LISTING (6 sequences)

SEQ ID NO: 1 - DNA, 777 bp, Artificial Sequence
  synthesized sequence
gctcctcttcctgatacaccgggagcgccatttcctgctgtcgcaaacttcgaccgcagcggcccttacactgtttctagccagtcagaagggccgagctgtcgcatctatagacctcgcgacctgggtcagggaggcgtacgccatccggttattctttggggcaacggcactggtgctggaccgtctacatatgcaggcttgctttcacactgggcaagccacggtttcgttgtagcggctgcggaaacatctaacgctggtaccggacgcgaaatgctcgcctgcctggactatctggtacgtgagaacgacaccccctacggcacctattccggcaagctcaataccgggcgagtcggcacttctgggcattctcaaggtggaggcgggtcaatcatggctggccaggatacgagagtacgtacaacggcgccgatccagccttacactcttggcctgggacacgacagcgcttctcaacgccgccaacagggaccgatgttccttatgtctggtggcggagacacaatcgctttcccttacctcaacgctcagccggtctaccgccgtgcaaacgtacctgtattctggggcgaaagacgttacgtttcacacttcgaaccggtaggtagcggtggggcttatcgcggcccgtctacagcatggttccgcttccaacttatggatgaccaagacgctcgcgctacattctacggcgcgcagtgcagcctttgcacttctttactttggtcagtcgaacgccgcgggctttaa

SEQ ID NO: 2 - protein, 258 aa, Pseudomonas mendocina
APLPDTPGAPFPAVANFDRSGPYTVSSQSEGPSCRIYRPRDLGQGGVRHPVILWGNGTGAGPSTYAGLLSHWASHGFVVAAAETSNAGTGREMLACLDYLVRENDTPYGTYSGKLNTGRVGTSGHSQGGGGSIMAGQDTRVRTTAPIQPYTLGLGHDSASQRRQQGPMFLMSGGGDTIAFPYLNAQPVYRRANVPVFWGERRYVSHFEPVGSGGAYRGPSTAWFRFQLMDDQDARATFYGAQCSLCTSLLWSVERRGL

SEQ ID NO: 3 - DNA, 608 bp, Bacillus subtilis
gaattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 4 - DNA, 87 bp, Bacillus subtilis
gtgagaagcaaaaaattgtggatcagcttgttgtttgcgttaacgttaatctttacgatggcgttcagcaacatgtctgcgcaggct

SEQ ID NO: 5 - DNA, 87 bp, Artificial Sequence
  synthesized sequence
gtgagaagcaaaaaattgtggatcagcttgttgtttgcgttaacgttagcggcttcttgcctgagcgtctgtgcaactgtagctgca

SEQ ID NO: 6 - DNA, 247 bp, Bacillus amyloliqufaciens
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

(19) United States
(12) Patent Application Publication
(10) Pub. No.: US 2024/0150738 A1
(43) Pub. Date: May 9, 2024

(54) VARIANT ENZYMES AND USES THEREOF

(71) Applicant: DANISCO US INC., PALO ALTO, CA (US)

(72) Inventors: CHRISTIAN D ADAMS, SAN FRANCISCO, CA (US); LILIA MARIA BABE, EMERALD HILLS, CA (US); GUO FENG, LOS ALTOS, CA (US); AMY DEMING LIU, SUNNYVALE, CA (US)

(21) Appl. No.: 18/280,544

(22) PCT Filed: Mar. 16, 2022
(86) PCT No.: PCT/US2022/020560

§ 371 Date: Sep. 6, 2023

Related U.S. Application Data
(60) Provisional application No. 63/162,132, filed on Mar. 17, 2021.

(51) Int. Cl. C12N 9/18; C11D 3/386; D06M 16/00; D06M 101/32

(52) U.S. Cl. CPC C12N 9/18; C11D 3/38627; D06M 16/003; C11D 2111/12; C12Y 301/01; D06M 2101/32

(57) ABSTRACT

The present disclosure relates to variant lipolytic enzymes, more particularly variant lipolytic enzymes that have improved stability and/or improved hydrolytic activity on a polyester. Such variant lipolytic enzymes find use in the degradation of polyesters, such as polyethylene terephthalate. Also provided are compositions and methods related to such variant lipolytic enzymes.

### CROSS REFERENCE

This application is a 371 of International Application No. PCT/US2022/020560, filed Mar. 16, 2022, and claims the benefit of U.S. Provisional Application No. 63/162,132, filed Mar. 17, 2021, which are incorporated by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named “20230906_NB41729USPCT_ST25” created on Sep. 6, 2023, and having a size of 5,655 bytes and is filed concurrently with the specification. The sequence listing contained in this ASCII formatted document is part of the specification and is herein incorporated by reference in its entirety.

The present disclosure relates to variant lipolytic enzymes, more particularly variant lipolytic enzymes that have improved stability and/or improved hydrolytic activity on a polyester. Such variant lipolytic enzymes find use in the degradation of polyesters, such as polyethylene terephthalate. Also provided are compositions and methods related to such variant lipolytic enzymes.

### BACKGROUND

Polyesters, such as polyethylene terephthalate (PET), are used in a large number of products and processes, such as in the manufacture of clothes, carpets, various packaging and plastics (e.g. automobile plastics), which has led to the accumulation of polyesters in landfills and may be an ecological problem.

Various enzymes, such as lipolytic enzymes, are able to catalyse the hydrolysis of a variety of polymers, including polyesters. Some of these enzymes are being investigated for use in a number of industrial applications, such as detergents for laundry and dishwashing applications, as degrading enzymes for processing biomass and food, as biocatalysts in detoxification of environmental pollutants or for the treatment of polyester fabrics in the textile industry. The use of such enzymes is of particular interest for hydrolysing polyesters, such as PET.

There is a continuing need for lipolytic enzymes with improved activity and/or improved stability that can be used in compositions for treating fabrics and/or textiles and for methods for degrading polyesters.

### SUMMARY

In one embodiment, the disclosure provides variant lipolytic enzymes comprising an amino acid sequence having at least 70% identity to the full length amino acid sequence of SEQ ID NO: 2, comprising one or more substitutions at positions selected from the group consisting of 14, 70, 117, 161, 175, 212, 226, 236, 239, 252, 254, 256, and 258, where the positions are numbered by reference to the amino acid sequence of SEQ ID NO: 2, and where the variant has hydrolytic activity on a polyester. In one embodiment, the disclosure provides variant lipolytic enzymes comprising one or more substitutions selected from the group consisting of V014S, R040A/T, G059Y, G061D, T064V, A066D, S070E, Q161H, G175A, R190L, S212D, F226L, A236P, Y239I, L249P, S252L, E254Q, R256K, and L258F, where the positions are numbered by reference to the amino acid sequence of SEQ ID NO: 2.

In another embodiment, the disclosure provides variant lipolytic enzymes comprising an amino acid sequence having at least 70% identity to the full length amino acid sequence of SEQ ID NO: 2, where the lipase variant comprises one, two, three, four, or more substitutions selected from the group consisting of V014S, R040A/T, G059Y, G061D, T064V, A066D, S070E, T117L, Q161H, G175A, T177N/R, I178L, F180P, Y182A/L, R190L, S205G, F207T, V210I, S212D, F226L, A236P, Y239I, L249P, S252L, E254Q, R256K, and L258F, where the positions are numbered by reference to the amino acid sequence of SEQ ID NO: 2, where the variant has esterase activity.

In yet another embodiment, variant lipolytic enzymes, or an active fragments thereof, are provided, where the variant lipolytic enzymes comprise:
(a) an amino acid sequence having at least 70% identity to the full-length amino acid sequence of SEQ ID NO: 2,
(b) two or more substitutions at positions selected from
   i) a substitution at position 61 and at least one additional substitution from a position selected from: 14, 40, 59, 64, 66, 70, 161, 175, 180, 182, 190, 205, 212, 226, 236, 239, 249, 252, 254, 256, and 258,
   ii) a substitution at position 180 and at least one additional substitution at a position selected from 14, 40, 59, 61, 64, 66, 70, 161, 175, 182, 190, 205, 212, 226, 236, 239, 249, 252, 254, 256, and 258;
   iii) a substitution at position 182 and at least one additional substitution at a position selected from 14, 40, 59, 61, 64, 66, 70, 161, 175, 180, 190, 205, 212, 226, 236, 239, 249, 252, 254, 256, and 258; or
   iv) a substitution at position 205 and at least one additional substitution at a position selected from 40, 59, 61, 64, 66, 70, 161, 180, 182, 190, 212, 226, 236, 239, 249, 252, 254, 256, and 258;
   where the positions are numbered by reference to the amino acid sequence of SEQ ID NO: 2, and where the variant lipolytic enzyme has esterase activity.

In still another embodiment, variant lipolytic enzymes, or an active fragments thereof, are provided, where the variant lipolytic enzymes comprise:
(a) an amino acid sequence having at least 70% identity to the full-length amino acid sequence of SEQ ID NO: 2,
(b) a combination of mutations selected from the group consisting of
   (i) a combination of mutations G061D-Y182A/L and at least one additional mutation selected from the group consisting of V014S, R040A/T, G059Y, T064V, A066D, S07E, T117L, Q161H, G175A, T177N/R, I178L, F180P, R190L, S205G, F207T, V210I, S212D, F226L, A236P, Y239I, L249P, S252I, E254Q, R256K, and L258F,
   (ii) a combination of mutations G061D-S205G and at least one additional mutation selected from the group consisting of V014S, R040A/T, G059Y, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
   (iii) a combination of mutations F180P-Y182A/L and at least one additional mutation selected from the group consisting of V014S, R040A/T, G059Y, G061D, T064V, A066D, S07E, T117L, Q161H, G175A, T177N/R, I178L, R190L, S205G, F207T, V210I, S212D, F226L, A236P, Y239I, L249P, S252I, E254Q, R256K, and L258F,
   (iv) a combination of mutations F180P-S205G and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F,
   (v) a combination of mutations Y182A/L-S205G and at least one additional mutation selected from the group consisting of V014S, R040A/T, G059Y, G061D, T064V, A066D, S07E, T117L, Q161H, G175A, T177N/R, I178L, F180P, R190L, F207T, V210I, S212D, F226L, A236P, Y239I, L249P, S252I, E254Q, R256K, and L258F,
   (vi) a combination of mutations V014S-R040A and at least one additional mutation selected from the group consisting of G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, R256K, and L258F,
   (vii) a combination of mutations V014S-R256K and at least one additional mutation selected from the group consisting of R040A/T, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
   (viii) a combination of mutations R040A-E254Q and at least one additional mutation selected from the group consisting of V014S, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
   (ix) a combination of mutations R040A-S205G and at least one additional mutation selected from the group consisting of V014S, G059Y, G061D, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
   (x) a combination of mutations G059Y-A236P and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, Y239I, L249P, S252I, E254Q, R256K, and L258F,
   (xi) a combination of mutations G059Y-E254Q and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
   (xii) a combination of mutations R040A-F180P and at least one additional mutation selected from the group consisting of V014S, G059Y, G061D, T064V, A066D, S07E, T117L, Q161H, G175A, T177N/R, I178L, Y182A/L, R190L, S205G, F207T, V210I, S212D, F226L, A236P, Y239I, L249P, S252I, E254Q, R256K, and L258F,
   (xiii) a combination of mutations G061D-F226L and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
   (xiv) a combination of mutations G061D-R256K and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
   (xv) a combination of mutations T064V-F226L and at least one additional mutation selected from the group consisting of V014S, R040A/T, G059Y, G061D, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
   (xvi) a combination of mutations A066D-F226L and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
   (xvii) a combination of mutations A066D-L249P and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, F226L, A236P, Y239I, S252I, E254Q, R256K, and L258F,
   (xviii) a combination of mutations S070E-L258F and at least one additional mutation selected from the group consisting of V014S, R040A/T, G059Y, G061D, T064V, A066D, T117L, Q161H, G175A, T177N/R, I178L, F180P, Y182A/L, R190L, S205G, F207T, V210I, S212D, F226L, A236P, Y239I, L249P, S252I, E254Q, and R256K,
   (xix) a combination of mutations T117L-S205G and at least one additional mutation selected from the group consisting of V014S, R040A/T, G059Y, G061D, T064V, A066D, S07E, Q161H, G175A, T177N/R, I178L, F180P, Y182A/L, R190L, F207T, V210I, S212D, F226L, A236P, Y239I, L249P, S252I, E254Q, R256K, and L258F,
   (xx) a combination of mutations T117L-L249P and at least one additional mutation selected from the group consisting of V014S, R040A/T, G059Y, G061D, T064V, A066D, S07E, Q161H, G175A, T177N/R, I178L, F180P, Y182A/L, R190L, S205G, F207T, V210I, S212D, F226L, A236P, Y239I, S252I, E254Q, R256K, and L258F,
   (xxi) a combination of mutations Y182A/L-L249P and at least one additional mutation selected from the group consisting of V014S, R040A/T, G059Y, G061D, T064V, A066D, S07E, T117L, Q161H, G175A, T177N/R, I178L, F180P, R190L, S205G, F207T, V210I, S212D, F226L, A236P, Y239I, S252I, E254Q, R256K, and L258F,
   (xxii) a combination of mutations Q161H-Y239I and at least one additional mutation selected from the group consisting of V014S, R040A/T, G059Y, G061D, T064V, A066D, S07E, T117L, G175A, T177N/R, I178L, F180P, Y182A/L, R190L, S205G, F207T, V210I, S212D, F226L, A236P, L249P, S252I, E254Q, R256K, and L258F, and
   (xxiii) a combination of mutations G175A-A236P and at least one additional mutation selected from the group consisting of V014S, R040A/T, G059Y, G061D, T064V, A066D, S07E, T117L, Q161H, T177N/R, I178L, F180P, Y182A/L, R190L, S205G, F207T, V210I, S212D, F226L, Y239I, L249P, S252I, E254Q, R256K, and L258F,

where the positions are numbered by reference to the amino acid sequence of SEQ ID NO: 2, and where the variant lipolytic enzyme has esterase activity.

In another embodiment, variant lipolytic enzymes, or an active fragments thereof, are provided, where the variant lipolytic enzymes comprise:
(a) an amino acid sequence having at least 70% identity to the full length amino acid sequence of SEQ ID NO: 2,
(b) a combination of mutations selected from the group consisting of:
   (i) T177N/R-F180P-S205G and at least one additional mutation selected from the group consisting of V014S, R040A/T, G059Y, G061D, T064V, A066D, S07E, T117L, Q161H, G175A, I178L, Y182A/L, R190L, F207T, V210I, S212D, F226L, A236P, Y239I, L249P, S252I, E254Q, R256K, and L258F;
   (ii) R040A/T-F180P-S205G and at least one additional mutation selected from the group consisting of V014S, G059Y, G061D, T064V, A066D, S07E, T117L, Q161H, G175A, T177N/R I178L, Y182A/L, R190L, F207T, V210L, S212D, F226L, A236P, Y239I, L249P, S252I, E254Q, R256K, and L258F;
   (iii) G059Y-F180P-S205G and at least one additional mutation selected from the group consisting of V014S, R040A/T, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
   (iv) G061D-F180P-S205G and at least one additional mutation selected from the group consisting of V014S, R040A/T, G059Y, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
   (v) T064V-F180P-S205G and at least one additional mutation selected from the group consisting of V014S, R040A/T, G059Y, G061D, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
   (vi) A066D-F180P-S205G and at least one additional mutation selected from the group consisting of V014S, R040A/T, G059Y, G061D, T064V, S07E, T117L, Q161H, G175A, T177N/R, I178L, Y182A/L, R190L, F207T, V210I, S212D, F226L, A236P, Y239I, L249P, S252I, E254Q, R256K, and L258F;
   (vii) S070E-F180P-S205G and at least one additional mutation selected from the group consisting of V014S, R040A/T, G059Y, G061D, T064V, A066D, T117L, Q161H, G175A, T177N/R, I178L, Y182A/L, R190L, F207T, V210I, S212D, F226L, A236P, Y239I, L249P, S252I, E254Q, R256K, and L258F;
   (viii) T117L-F180P-S205G and at least one additional mutation selected from the group consisting of V014S, R040A/T, G059Y, G061D, T064V, A066D, S07E, Q161H, G175A, T177N/R, I178L, Y182A/L, R190L, F207T, V210I, S212D, F226L, A236P, Y239I, L249P, S252I, E254Q, R256K, and L258F;
   (ix) Q161H-F180P-S205G and at least one additional mutation selected from the group consisting of V014S, R040A/T, G059Y, G061D, T064V, A066D, S07E, T117L, G175A, T177N/R, I178L, Y182A/L, R190L, F207T, V210I, S212D, F226L, A236P, Y239I, L249P, S252I, E254Q, R256K, and L258F;
   (x) I178L-F180P-S205G and at least one additional mutation selected from the group consisting of V014S, R040A/T, G059Y, G061D, T064V, A066D, S07E, T117L, Q161H, G175A, T177N/R, Y182A/L, R190L, F207T, V210I, S212D, F226L, A236P, Y239I, L249P, S252I, E254Q, R256K, and L258F;
   (xi) F180P-Y182A/L-S205G and at least one additional mutation selected from the group consisting of V014S, R040A/T, G059Y, G061D, T064V, A066D, S07E, T117L, Q161H, G175A, T177N/R, I178L, R190L, F207T, V210L, S212D, F226L, A236P, Y239I, L249P, S252I, E254Q, R256K, and L258F;
   (xii) F180P-R190L-S205G and at least one additional mutation selected from the group consisting of V014S, R040A/T, G059Y, G061D, T064V, A066D, S07E, T117L, Q161H, G175A, T177N/R, I178L, Y182A/L, F207T, V210I, S212D, F226L, A236P, Y239I, L249P, S252I, E254Q, R256K, and L258F;
   (xiii) F180P-S205G-F207T and at least one additional mutation selected from the group consisting of V014S, R040A/T, G059Y, G061D, T064V, A066D, S07E, T117L, Q161H, G175A, T177N/R, I178L, Y182A/L, R190L, V210I, S212D, F226L, A236P, Y239I, L249P, S252I, E254Q, R256K, and L258F;
   (xiv) F180P-S205G-V210I and at least one additional mutation selected from the group consisting of V014S, R040A/T, G059Y, G061D, T064V, A066D, S07E, T117L, Q161H, G175A, T177N/R, I178L, Y182A/L, R190L, F207T, S212D, F226L, A236P, Y239I, L249P, S252I, E254Q, R256K, and L258F;
   (xv) F180P-S205G-S212D and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, F226L, A236P, Y239I, L249P, S252I, E254Q, R256K, and L258F;
   (xvi) F180P-S205G-F226L and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, A236P, Y239I, L249P, S252I, E254Q, R256K, and L258F;
   (xvii) F180P-S205G-A236P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, Y239I, L249P, S252I, E254Q, R256K, and L258F;
   (xviii) F180P-S205G-Y239I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, L249P, S252I, E254Q, R256K, and L258F;
   (xix) F180P-S205G-L249P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, S252I, E254Q, R256K, and L258F;
   (xx) F180P-S205G-S252I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, E254Q, R256K, and L258F;
   (xxi) F180P-S205G-E254Q and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, S252I, R256K, and L258F;
   (xxii) F180P-S205G-R256K and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and L258F; and
   (xxiii) F180P-S205G-L258F and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and R256K where the positions are numbered by reference to the amino acid sequence of SEQ ID NO: 2, and where the variant lipolytic enzyme has esterase activity.

Also provided herein are polynucleotides encoding such variant lipolytic enzymes, or fragments thereof, as well as expression vectors and recombinant host cells.

In addition, enzyme compositions are provided comprising a variant lipolytic enzyme as disclosed herein.

In another embodiment, the disclosure provides cleaning compositions or detergent compositions comprising a variant lipolytic enzyme as disclosed herein and at least one adjunct selected from the group consisting of surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

In another embodiment, fabric treatment compositions are provided comprises a variant lipolytic enzyme as disclosed herein and at least one adjunct selected from the group consisting of surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

Also provided are methods for treating a fabric or textile comprising, (i) contacting a fabric or textile with a variant lipolytic enzyme as disclosed herein or a composition comprising said variant lipolytic enzyme, and (ii) optionally, rinsing the fabric or textile.

Also provided are methods for degrading a polyester or a polyester containing material as well as methods for the enzymatic depolymerization of a polyester or a polyester containing material.

### FIGURES

FIG. 1 provides a graphic representation of concentration dependent polyesterase activity of variant lipolytic enzymes compared to a reference, wild-type lipolytic enzyme in various detergent compositions. FIG. 1A: provides concentration dependent polyesterase activity of P. mendocina WT lipase and variants PEV001, PEV017 and PEV132 measured in Formula A HDL detergent. FIG. 1B: Provides concentration dependent polyesterase activity of P. mendocina WT lipase and variants PEV001, PEV017 and PEV132 measured in PNB detergent.

FIG. 1C: Provides concentration dependent polyesterase activity of P. mendocina WT lipase and variants PEV001, PEV017 and PEV132 measured in Tide HDL detergent. FIG. 1D: Provides concentration dependent polyesterase activity of P. mendocina WT lipase and variants PEV001, PEV017 and PEV132 measured in Test HDL 1 detergent.

FIG. 2 provides residual activity measurements of various embodiments of the present disclosure. Samples of Pseudomonas mendocina lipase WT, PEV001, PEV017 and PEV132 were heat stressed at 50° C. for 19 hours, and residual enzyme activity was measured using pNB assay.

FIG. 3 provides dose response curves for polyester activity on swatches for cutinases PEV017, PEV132.

FIG. 4 Depicts a model of P. mendocina WT lipase with docked substrate 2-HE(MHET)₃ using the PDB 2FX5 structure highlighting the catalytic residues.

FIG. 5 Depicts a model of substitutions near P. mendocina lipase catalytic residues and substrate binding domain that benefit PET activity.

FIG. 6 Depicts a model of substitutions that improve P. mendocina lipase thermostability in detergents.

FIG. 7 Depicts a structure superposition of P. mendocina WT lipase (PDB: 2FX5) and LCC (leaf-branch compost cutinase, PDB: 4EB0).

### DESCRIPTION

The present disclosure provides variant lipolytic enzymes, compositions (e.g. enzyme and detergent compositions) comprising such variant lipolytic enzymes, and methods using such variant lipolytic enzymes and compositions, for example, for washing or treating textiles and/or fabrics, and the degradation of polyesters.

Prior to describing embodiments of present compositions and methods, the following terms are defined.

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the specification as a whole. Also, as used herein, the singular terms “a,” “an,” and “the” include the plural reference unless the context clearly indicates otherwise. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein, the term “polymer” refers to a chemical compound or mixture of compounds whose structure is constituted of multiple repeating units linked by covalent chemical bonds. Within the context of the disclosure, the term polymer includes natural or synthetic polymers, constituting of a single type of repeat unit (i.e., homopolymers) or of a mixture of different repeat units (i.e., block copolymers and random copolymers).

The term “polyester-containing material” or “polyester-containing product”, as used herein, refer to a product, such as a textile, fabric, or plastic product, comprising at least one polyester in crystalline, semi-crystalline, or substantially amorphous forms. In some embodiments, the polyester-containing material refers to any item made from at least one plastic material, such as plastic sheet, tube, rod, profile, shape, film, block, etc., which contains at least one polyester, and possibly other substances or additives, such as plasticizers, mineral or organic fillers. In some embodiments, the polyester-containing material refers to a plastic compound, or plastic formulation, in a molten or solid state, suitable for making a plastic product. In some embodiments, the polyester-containing material refers to a textile or fabric or fibers comprising at least one polyester. In some embodiments, the polyester-containing material refers to plastic waste or fiber waste comprising at least one polyester.

As used herein, the term “polyester” refers to polymers where the monomers are bonded by ester linkages. As used herein, the term “polyester” includes, but is not limited to, those polyesters selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

The term “fabric” refers to, for example, woven, knit, and non-woven material, as well as staple fibers and filaments that can be converted to, for example, yarns and woven, knit, and non-woven fabrics. The term encompasses material made from natural, as well as synthetic (e.g., manufactured) fibers, and combinations thereof.

The term “textile”, as used herein, refers to any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and toweling. The textile may include cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well. In the context of the present application, the term “textile” is used interchangeably with fabric and cloth. In some embodiments, textiles include those materials that include at least one polyester.

The term “laundering” includes both household laundering and industrial laundering and means the process of treating textiles with a solution containing a cleaning or detergent composition as provided herein. The laundering process can for example be carried out using e.g. a household or an industrial washing machine or can be carried out by hand.

The term “wash cycle” refers to a washing operation in which textiles are immersed in a wash liquor, mechanical action of some kind is applied to the textile to release stains or to facilitate flow of wash liquor in and out of the textile and finally the superfluous wash liquor is removed. After one or more wash cycles, the textile is generally rinsed and dried.

The term “wash liquor” is defined herein as the solution or mixture of water and detergent components optionally including variant lipolytic enzymes as provided herein.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, the term “variant polypeptide” refers to a polypeptide comprising an amino acid sequence that differs in at least one amino acid residue from the amino acid sequence of a parent or reference polypeptide (including but not limited to wild-type polypeptides).

### Variant Lipolytic Enzymes

In one embodiment, variant lipolytic enzymes are provided. In some embodiments, the variant lipolytic enzymes provided herein have hydrolytic activity on at least one polyester.

As used herein, a lipolytic enzyme includes an enzyme, polypeptide, or protein exhibiting a lipid degrading capability such as a capability of degrading a triglyceride or a phospholipid. The lipolytic enzyme can be, for example, a lipase, a phospholipase, an esterase or a cutinase. Lipolytic enzymes can be enzymes having a/s hydrolase fold. These enzymes typically have a catalytic triad of serine, aspartic acid and histidine residues. The a/s hydrolases include lipases and cutinases. Cutinases show little, if any, interfacial activation, where lipases often undergo a conformational change in the presence of a lipid-water interface (Longhi and Cambillau (1999) Biochimica et Biophysica Acta 1441:185-96). An active fragment of a lipolytic enzyme is a portion of a lipolytic enzyme that retains a lipid degrading capability. An active fragment retains the catalytic triad. As used herein, lipolytic activity can be determined according to any procedure known in the art (see, e.g., Gupta et al., Biotechnol. Appl. Biochem., 37:63-71, 2003; U.S. Pat. No. 5,990,069; and International Patent Publication No. WO 96/18729A1).

In some embodiments, lipolytic enzymes of the present disclosure are a/0 hydrolases. In some embodiments, lipolytic enzymes of the present disclosure are lipases. In some embodiments, lipolytic enzymes of the present disclosure are cutinases. In some embodiments, lipolytic enzymes of the present disclosure are esterases.

In some embodiments, lipolytic enzymes of the present disclosure are alpha/beta hydrolases. In some embodiments, lipolytic enzymes of the present disclosure are lipases. In some embodiments, lipolytic enzymes of the present disclosure are cutinases. In some embodiments, lipolytic enzymes of the present disclosure are polyesterases.

As used herein, a “a carboxylic ester hydrolase” (E.C. 3.1.1) refers to an enzyme that acts on carboxylic acid esters.

As used herein, a “lipase”, “lipase enzyme”, “lipolytic enzymes”, “lipolytic polypeptides”, or “lipolytic proteins” refers to an enzyme, polypeptide, or protein exhibiting a lipid degrading capability such as a capability of degrading a triglyceride or a phospholipid. The lipolytic enzyme may be, for example, a lipase, a phospholipase, an esterase, a polyesterase, or a cutinase. As used herein, lipolytic activity may be determined according to any procedure known in the art (see, e.g., Gupta et al, Biotechnol. Appl. Biochem., 37:63-71, 2003; U.S. Pat. No. 5,990,069; and International Patent Publication No. WO 96/1 8729A1). In one embodiment, lipolytic activity can be determined on 4-nitrophenyl butyrate (pNB) as provided in Example 2.

A used herein, “cutinase” refers to lipolytic enzymes capable of hydrolyzing cutin substrates.

Cutinases include those derived from various fungi and from bacterial sources. Cutinases include those described in P. E. Kolattukudy, “Lipases”, Ed. B Borgstrom and H. L. Brockman, Elsevier 1984, 471-504; S. Longhi et al., J. of Molecular Biology, 268 (4), 779-799 (1997); U.S. Pat. No. 5,827,719; WO 94/14963; WO 94/14964; WO 00/05389; Appl. Environm. Microbiol 64, 2794-2799, 1998; Proteins: Structure, Function and Genetics 26, 442-458, 1996; J. of Computational Chemistry 17, 1783-1803, 1996; Protein Engineering 6, 157-165, 1993. Cutinases may be naturally occurring or genetically modified cutinase obtained by UV irradiation, N-methyl-N′-nitrosoguanidine (NTG) treatment, ethyl methanesulfonate (EMS) treatment, nitrous acid treatment, acridine treatment or the like, recombinant strains induced by the genetic engineering procedures such as cell fusion and gene recombination and so forth.

As used herein, the term “polyesterase” or “PETase” refers to an enzyme that has significant capability to catalyze the hydrolysis and/or surface modification of polyester. Suitable polyesterases may be isolated from animal, plant, fungal and bacterial sources. The aforementioned microorganisms may be, in addition to being isolated from wild strains, may be isolated from any of mutant strains obtained by UV irradiation, N-methyl-N′-nitrosoguanidine (NTG) treatment, ethyl methanesulfonate (EMS) treatment, nitrous acid treatment, acridine treatment or the like, recombinant strains induced by the genetic engineering procedures such as cell fusion and gene recombination and so forth. The polyesterase may catalyze the hydrolysis and/or surface modification of a polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

As used herein, “% identity or percent identity” refers to sequence similarity. Percent identity may be determined using standard techniques known in the art (See e.g., Smith and Waterman, Adv. Appl. Math. 2:482 [1981]; Needleman and Wunsch, J. Mol. Biol. 48:443 [1970]; Pearson and Lipman, Proc. Natl. Acad. Sci. USA 85:2444 [1988]; software programs such as GAP, BESTFIT, FASTA, and TFASTA in the Wisconsin Genetics Software Package (Genetics Computer Group, Madison, WI); and Devereux et al., Nucl. Acid Res. 12:387-395 [1984]). One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (See, Feng and Doolittle, J. Mol. Evol. 35:351-360 [1987]). The method is similar to that described by Higgins and Sharp (See, Higgins and Sharp, CABIOS 5:151-153 [1989]). Useful PILEUP parameters include a default gap weight of 3.00, a default gap length weight of 0.10, and weighted end gaps. Other useful algorithm is the BLAST algorithms described by Altschul et al., (See, Altschul et al., J. Mol. Biol. 215:403-410 [1990]; and Karlin and Altschul, Proc. Natl. Acad. Sci. USA 90:5873-5787 [1993]). The BLAST program uses several search parameters, most of which are set to the default values.

As used herein, “homologous proteins”, “homologs” or “homologous proteins” refers to proteins that have distinct similarity in primary, secondary, and/or tertiary structure. Protein homology can refer to the similarity in linear amino acid sequence when proteins are aligned. Homology can be determined by amino acid sequence alignment, e.g., using a program such as BLAST, MUSCLE, or CLUSTAL. Homologous search of protein sequences can be done using BLASTP and PSI-BLAST from NCBI BLAST with threshold (E-value cut-off) at 0.001. (Altschul et al., “Gapped BLAST and PSI BLAST a new generation of protein database search programs”, Nucleic Acids Res, Set 1; 25(17):3389-402(1997)). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997 and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. Using this information, protein sequences can be grouped and/or a phylogenetic tree built therefrom. Amino acid sequences can be entered in a program such as the Vector NTI Advance suite and a Guide Tree can be created using the Neighbor Joining (NJ) method (Saitou and Nei, Mol Biol Evol, 4:406-425, 1987). The tree construction can be calculated using Kimura's correction for sequence distance and ignoring positions with gaps. A program such as AlignX can display the calculated distance values in parentheses following the molecule name displayed on the phylogenetic tree.

A percent (%) amino acid sequence identity value is determined by the number of matching identical residues divided by the total number of residues of the “reference” sequence including any gaps created by the program for optimal/maximum alignment. If a sequence is 90% identical to SEQ ID NO: A, SEQ ID NO: A is the “reference” sequence. BLAST algorithms refer the “reference” sequence as “query” sequence.

The CLUSTAL W algorithm is another example of a sequence alignment algorithm (See, Thompson et al., Nucleic Acids Res, 22:4673-4680, 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues×100) relative to the “reference” polypeptide. Such a variant would be encompassed by a variant having “at least 99% sequence identity” to the polypeptide.

In some embodiments, the variant lipase includes those derived from 2FX5_A, and those derived from the lipase disclosed in WO88/09367, U.S. Pat. Nos. 5,512,203, 5,389,536, U.S. Patent Publication No. US2003199068, European Patent Publication No. EP1543117, and WO 03/076580.

In some embodiments, the variant lipolytic enzymes provided herein comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 2. In some embodiments, the variant lipolytic enzymes have an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 2 and has esterase activity.

The disclosure provides variant lipolytic enzymes or an active fragment thereof, comprising an amino acid sequence having at least 70% identity to the full length amino acid sequence of SEQ ID NO: 2, comprising two, three, four, or more substitutions at positions selected from the group consisting of 14, 40, 59, 61, 64, 66, 70, 117, 161, 177, 178, 180, 182, 190, 205, 207, 210, 212, 226, 236, 239, 249, 252, 254, 256, and 258, wherein the positions are numbered by reference to the amino acid sequence of SEQ ID NO: 2. In some embodiments, the variant lipolytic enzyme has esterase activity.

In some embodiments, the variant lipolytic enzymes provided herein comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 comprising one or more substitutions at positions selected from the group consisting of 14, 70, 117, 161, 175, 212, 226, 236, 239, 252, 254, 256, and 258, where the positions are numbered by reference to the amino acid sequence of SEQ ID NO: 2. In some embodiments, the variant lipolytic enzyme may further comprise at least one additional substitution at a position selected from 40, 59, 61, 64, 66, 177, 178, 180, 182, 190, 205, 207, 210, and 249, where the positions are numbered by reference to the amino acid sequence of SEQ ID NO: 2. In some embodiments, such variant lipolytic enzymes have esterase activity.

In some embodiments, the variant lipolytic enzymes provided herein comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 comprising two or more substitutions at positions selected from
i) a substitution at position 61 and at least one additional substitution from a position selected from: 14, 40, 59, 64, 66, 70, 117, 161, 175, 177, 178, 180, 182, 190, 205, 207, 210, 212, 226, 236, 239, 249, 252, 254, 256, and 258,
ii) a substitution at position 180 and at least one additional substitution at a position selected from 14, 40, 59, 61, 64, 66, 70, 117, 161, 175, 177, 178, 182, 190, 205, 207, 210, 212, 226, 236, 239, 249, 252, 254, 256, and 258;
iii) a substitution at position 182 and at least one additional substitution at a position selected from 14, 40, 59, 61, 64, 66, 70, 117, 161, 175, 177, 178, 180, 190, 205, 207, 210, 212, 226, 236, 239, 249, 252, 254, 256, and 258; or
iv) a substitution at position 205 and at least one additional substitution at a position selected from 40, 59, 61, 64, 66, 70, 117, 161, 177, 178, 180, 182, 190, 207, 210, 212, 226, 236, 239, 249, 252, 254, 256, and 258; where the positions are numbered by reference to the amino acid sequence of SEQ ID NO: 2. In some embodiments, such variant lipolytic enzymes have esterase activity.

In some embodiments, the disclosure provides a variant lipolytic enzyme as provided herein comprises an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 comprising one or more substitutions selected from the group consisting of V014S, R040A/T, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, R256K, and L258F, where the positions are numbered by reference to the amino acid sequence of SEQ ID NO: 2. In some embodiments, such variant lipolytic enzymes have esterase activity.

In some embodiments, the variant lipolytic enzyme provided herein comprises an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 and comprises one, two, three, four, five, or more substitutions, or combination of substitutions selected from the group consisting of R040A, G059Y, G061D, T064V, A066D, S07E, Q161H, F180P, Y182A, R190L, S205G, S212D, F226L, A236P, Y239I, L249P, S252I, E254Q, R256K, L258F, G061D-Y182A, G061D-S205G, F180P-Y182A, F180P-S205G, Y182A-S205G, V014S-R040A, V014S-R256K, R040A-E254Q, R040A-S205G, G059Y-A236P, G059Y-E254Q, R040A-F180P, G061D-F226L, G061D-R256K, T064V-F226L, A066D-F226L, A066D-L249P, S070E-L258F, T117L-S205G, T117L-L249P, Y182A-L249P, Q161H-Y239I, G175A-A236P, F180P-S205G, F180P-S252I, Y182A-S212D, Y182A-L258F, R190L-S205G, R190L-L249P, S205G-F207T, S205G-F226L, F207T-R256K, S212D-L249P, S212D-E254Q, F226L-R256K, Y239I-L249P, V014S-R040A-F180P, V014S-S205G-R256K, R040A-F226L-E254Q, G059Y-T117L-A236P, G059Y-A236P-E254Q, G059Y-L249P-L258F, G059Y-G061D-F226L, G061D-R256K-L258F, T064V-F226L-S252I, A066D-F226L-R256K, A066D-Y239I-L249P, S070E-S212D-L258F, T117L-S205G-F226L, T117L-F226L-A236P, T117L-S205G-S252I, Q161H-Y182A-A236P, Q161H-Y239I-L249P, G059Y-G175A-F180P, G175A-A236P-L258F, T064V-T177R-F180P, F180P-S205G-Y239I, R040A-F180P-S252I, T117L-Y182A-S212D, Y182A-F226L-L258F, T117L-R190L-L249P, S205G-F207T-A236P, S205G-F226L-L249P, G059Y-F207T-R256K, T064V-S212D-L249P, R040A-S212D-E254Q, F226L-S252I-R256K, Y239I-L249P-R256K, T177R-F180P-S205G, R040A-F180P-S205G, T064V-F180P-S205G, F180P-S205G-L249P, F180P-R190L-S205G, G061D-Y182A-S205G, F180P-Y182A-S205G, G061D-F180P-Y182A-S205G, and G061D-T117L-I178L-F180P-Y182A-R190L-S205G-F207T-S212D-F226L-Y239I-L249P-S252I-L258F, where the positions are numbered by reference to the amino acid sequence of SEQ ID NO: 2. In some embodiments, such variant lipolytic enzymes have esterase activity.

In some embodiments, variant lipolytic enzymes are provided herein comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 and comprises three, four, five, six, seven, eight, or more substitutions selected from:
(i) one, two, three, or four amino acid substitutions selected from the group consisting of R40A/T, T177R/N, F180P, and S205G;
(ii) one, two, or three or more additional substitutions selected from the group consisting of T64V, Y182L, and R190L, and
(iii) at least one additional amino acid substitution selected from the group consisting of G059Y, G061D, A066D, S07E, Q161H, S212D, F226L, A236P, Y239I, L249P, S252I, E254Q, R256K, and L258F, where the positions are numbered by reference to the amino acid sequence of SEQ ID NO: 2. In some embodiments, such variant lipolytic enzymes have esterase activity.

In some embodiments, variant lipolytic enzymes are provided herein comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 and comprises a combination of substitutions selected from:
(i) a combination of mutations G061D-Y182A/L and at least one additional mutation selected from the group consisting of V014S, R040A/T, G059Y, T064V, A066D, S07E, T117L, Q161H, G175A, T177N/R, I178L, F180P, R190L, S205G, F207T, V210I, S212D, F226L, A236P, Y239I, L249P, S252I, E254Q, R256K, and L258F,
(ii) a combination of mutations G061D-S205G and at least one additional mutation selected from the group consisting of V014S, R040A/T, G059Y, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
(iii) a combination of mutations F180P-Y182A/L and at least one additional mutation selected from the group consisting of V014S, R040A/T, G059Y, G061D, T064V, A066D, S07E, T117L, Q161H, G175A, T177N/R, I178L, R190L, S205G, F207T, V210I, S212D, F226L, A236P, Y239I, L249P, S252I, E254Q, R256K, and L258F,
(iv) a combination of mutations F180P-S205G and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F,
(v) a combination of mutations Y182A/L-S205G and at least one additional mutation selected from the group consisting of V014S, R040A/T, G059Y, G061D, T064V, A066D, S07E, T117L, Q161H, G175A, T177N/R, I178L, F180P, R190L, F207T, V210I, S212D, F226L, A236P, Y239I, L249P, S252I, E254Q, R256K, and L258F,
(vi) a combination of mutations V014S-R040A and at least one additional mutation selected from the group consisting of G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, R256K, and L258F,
(vii) a combination of mutations V014S-R256K and at least one additional mutation selected from the group consisting of R040A/T, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
(viii) a combination of mutations R040A-E254Q and at least one additional mutation selected from the group consisting of V014S, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
(ix) a combination of mutations R040A-S205G and at least one additional mutation selected from the group consisting of V014S, G059Y, G061D, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
(x) a combination of mutations G059Y-A236P and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, Y239I, L249P, S252I, E254Q, R256K, and L258F,
(xi) a combination of mutations G059Y-E254Q and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
(xii) a combination of mutations R040A-F180P and at least one additional mutation selected from the group consisting of V014S, G059Y, G061D, T064V, A066D, S07E, T117L, Q161H, G175A, T177N/R, I178L, Y182A/L, R190L, S205G, F207T, V210I, S212D, F226L, A236P, Y239I, L249P, S252I, E254Q, R256K, and L258F,
(xiii) a combination of mutations G061D-F226L and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
(xiv) a combination of mutations G061D-R256K and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
(xv) a combination of mutations T064V-F226L and at least one additional mutation selected from the group consisting of V014S, R040A/T, G059Y, G061D, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
(xvi) a combination of mutations A066D-F226L and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
(xvii) a combination of mutations A066D-L249P and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, F226L, A236P, Y239I, S252I, E254Q, R256K, and L258F,
(xviii) a combination of mutations S070E-L258F and at least one additional mutation selected from the group consisting of V014S, R040A/T, G059Y, G061D, T064V, A066D, T117L, Q161H, G175A, T177N/R, I178L, F180P, Y182A/L, R190L, S205G, F207T, V210I, S212D, F226L, A236P, Y239I, L249P, S252I, E254Q, and R256K,
(xix) a combination of mutations T117L-S205G and at least one additional mutation selected from the group consisting of V014S, R040A/T, G059Y, G061D, T064V, A066D, S07E, Q161H, G175A, T177N/R, I178L, F180P, Y182A/L, R190L, F207T, V210I, S212D, F226L, A236P, Y239I, L249P, S252I, E254Q, R256K, and L258F,
(xx) a combination of mutations T117L-L249P and at least one additional mutation selected from the group consisting of V014S, R040A/T, G059Y, G061D, T064V, A066D, S07E, Q161H, G175A, T177N/R, I178L, F180P, Y182A/L, R190L, S205G, F207T, V210I, S212D, F226L, A236P, Y239I, S252I, E254Q, R256K, and L258F,
(xxi) a combination of mutations Y182A/L-L249P and at least one additional mutation selected from the group consisting of V014S, R040A/T, G059Y, G061D, T064V, A066D, S07E, T117L, Q161H, G175A, T177N/R, I178L, F180P, R190L, S205G, F207T, V210I, S212D, F226L, A236P, Y239I, S252I, E254Q, R256K, and L258F,
(xxii) a combination of mutations Q161H-Y239I and at least one additional mutation selected from the group consisting of V014S, R040A/T, G059Y, G061D, T064V, A066D, S07E, T117L, G175A, T177N/R, I178L, F180P, Y182A/L, R190L, S205G, F207T, V210I, S212D, F226L, A236P, L249P, S252I, E254Q, R256K, and L258F, and
(xxiii) a combination of mutations G175A-A236P and at least one additional mutation selected from the group consisting of V014S, R040A/T, G059Y, G061D, T064V, A066D, S07E, T117L, Q161H, T177N/R, I178L, F180P, Y182A/L, R190L, S205G, F207T, V210I, S212D, F226L, Y239I, L249P, S252I, E254Q, R256K, and L258F,
   where the positions are numbered by reference to the amino acid sequence of SEQ ID NO: 2. In some embodiments, such variant lipolytic enzymes have esterase activity.

In some embodiments, variant lipolytic enzymes are provided herein comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 and comprises a combination of mutations selected from the group consisting of
(i) T177N/R-F180P-S205G and at least one additional mutation selected from the group consisting of V014S, R040A/T, G059Y, G061D, T064V, A066D, S07E, T117L, Q161H, G175A, I178L, Y182A/L, R190L, F207T, V210I, S212D, F226L, A236P, Y239I, L249P, S252I, E254Q, R256K, and L258F;
(ii) R040A/T-F180P-S205G and at least one additional mutation selected from the group consisting of V014S, G059Y, G061D, T064V, A066D, S07E, T117L, Q161H, G175A, T177N/R I178L, Y182A/L, R190L, F207T, V210I, S212D, F226L, A236P, Y239I, L249P, S252I, E254Q, R256K, and L258F;
(iii) G059Y-F180P-S205G and at least one additional mutation selected from the group consisting of V014S, R040A/T, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
(iv) G061D-F180P-S205G and at least one additional mutation selected from the group consisting of V014S, R040A/T, G059Y, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
(v) T064V-F180P-S205G and at least one additional mutation selected from the group consisting of V014S, R040A/T, G059Y, G061D, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
(vi) A066D-F180P-S205G and at least one additional mutation selected from the group consisting of V014S, R040A/T, G059Y, G061D, T064V, S07E, T117L, Q161H, G175A, T177N/R, I178L, Y182A/L, R190L, F207T, V210I, S212D, F226L, A236P, Y239I, L249P, S252I, E254Q, R256K, and L258F;
(vii) S070E-F180P-S205G and at least one additional mutation selected from the group consisting of V014S, R040A/T, G059Y, G061D, T064V, A066D, T117L, Q161H, G175A, T177N/R, I178L, Y182A/L, R190L, F207T, V210I, S212D, F226L, A236P, Y239I, L249P, S252I, E254Q, R256K, and L258F;
(viii) T117L-F180P-S205G and at least one additional mutation selected from the group consisting of V014S, R040A/T, G059Y, G061D, T064V, A066D, S07E, Q161H, G175A, T177N/R, I178L, Y182A/L, R190L, F207T, V210I, S212D, F226L, A236P, Y239I, L249P, S252I, E254Q, R256K, and L258F;
(ix) Q161H-F180P-S205G and at least one additional mutation selected from the group consisting of V014S, R040A/T, G059Y, G061D, T064V, A066D, S07E, T117L, G175A, T177N/R, I178L, Y182A/L, R190L, F207T, V210I, S212D, F226L, A236P, Y239I, L249P, S252I, E254Q, R256K, and L258F;
(x) I178L-F180P-S205G and at least one additional mutation selected from the group consisting of V014S, R040A/T, G059Y, G061D, T064V, A066D, S07E, T117L, Q161H, G175A, T177N/R, Y182A/L, R190L, F207T, V210I, S212D, F226L, A236P, Y239I, L249P, S252I, E254Q, R256K, and L258F;
(xi) F180P-Y182A/L-S205G and at least one additional mutation selected from the group consisting of V014S, R040A/T, G059Y, G061D, T064V, A066D, S07E, T117L, Q161H, G175A, T177N/R, I178L, R190L, F207T, V210L, S212D, F226L, A236P, Y239I, L249P, S252I, E254Q, R256K, and L258F;
(xii) F180P-R190L-S205G and at least one additional mutation selected from the group consisting of V014S, R040A/T, G059Y, G061D, T064V, A066D, S07E, T117L, Q161H, G175A, T177N/R, I178L, Y182A/L, F207T, V210I, S212D, F226L, A236P, Y239I, L249P, S252I, E254Q, R256K, and L258F;
(xiii) F180P-S205G-F207T and at least one additional mutation selected from the group consisting of V014S, R040A/T, G059Y, G061D, T064V, A066D, S07E, T117L, Q161H, G175A, T177N/R, I178L, Y182A/L, R190L, V210I, S212D, F226L, A236P, Y239I, L249P, S252I, E254Q, R256K, and L258F;
(xiv) F180P-S205G-V210I and at least one additional mutation selected from the group consisting of V014S, R040A/T, G059Y, G061D, T064V, A066D, S07E, T117L, Q161H, G175A, T177N/R, I178L, Y182A/L, R190L, F207T, S212D, F226L, A236P, Y239I, L249P, S252I, E254Q, R256K, and L258F;
(xv) F180P-S205G-S212D and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, F226L, A236P, Y239I, L249P, S252I, E254Q, R256K, and L258F;
(xvi) F180P-S205G-F226L and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, A236P, Y239I, L249P, S252I, E254Q, R256K, and L258F;
(xvii) F180P-S205G-A236P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, Y239I, L249P, S252I, E254Q, R256K, and L258F;
(xviii) F180P-S205G-Y239I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, L249P, S252I, E254Q, R256K, and L258F;
(xix) F180P-S205G-L249P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, S252I, E254Q, R256K, and L258F;
(xx) F180P-S205G-S252I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, E254Q, R256K, and L258F;
(xxi) F180P-S205G-E254Q and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, S252I, R256K, and L258F;
(xxii) F180P-S205G-R256K and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and L258F; and
(xxiii) F180P-S205G-L258F and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and R256K,
   where the positions are numbered by reference to the amino acid sequence of SEQ ID NO: 2. In some embodiments, such variant lipolytic enzymes have esterase activity.

In some embodiments, the variant lipolytic enzymes provided herein comprise an amino acid sequence having at least 50%, 55%, 60%, 65%, 70%, 75%, 80%, 85%, 90%, 91%, 92%, 93%, 94%, 95%, 96%, 97%, 98%, 99% or 100% sequence identity to the full length amino acid sequence of SEQ ID NO: 2 and comprises a combination of mutations selected from the group consisting of R040T-T064V-T177N-F180P-Y182L-R190L-S205G-L249P, and R040T-T064V-T117L-T177N-I178L-F180P-Y182A-R190L-S205G-F207T-S212D-F226L-Y239I-L249P-S252I-L258F, where the positions are numbered by reference to the amino acid sequence of SEQ ID NO: 2.

In some embodiments, the variant lipolytic enzymes provided herein have esterase activity (e.g. ability to catalyze the hydrolysis and/or surface modification) on at least one polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof. In one embodiment, the variant lipolytic enzymes provided herein have esterase activity on PET.

Described herein is one or more isolated, non-naturally occurring, or recombinant polynucleotide comprising a nucleic acid sequence that encodes one or more variant lipolytic enzyme described herein, or recombinant polypeptide or active fragment thereof. One or more nucleic acid sequence described herein is useful in recombinant production (e.g., expression) of one or more variant lipolytic enzyme described herein, typically through expression of a plasmid expression vector comprising a sequence encoding the one or more variant lipolytic enzyme described herein or fragment thereof. One embodiment provides nucleic acids encoding one or more variant lipolytic enzyme described herein, wherein the variant is a mature form having lipolytic activity. In some embodiments, one or more variant lipolytic enzyme described herein is expressed recombinantly with a homologous pro-peptide sequence. In other embodiments, one or more variant lipolytic enzyme described herein is expressed recombinantly with a heterologous pro-peptide sequence.

One or more nucleic acid sequence described herein can be generated by using any suitable synthesis, manipulation, and/or isolation techniques, or combinations thereof. For example, one or more polynucleotide described herein may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the one or more polynucleotide described herein can be also facilitated by any suitable method known in the art, including but not limited to chemical synthesis using the classical phosphoramidite method (See e.g., Beaucage et al. Tetrahedron Letters 22:1859-69 (1981)), or the method described in Matthes et al., EMBO J. 3:801-805 (1984) as is typically practiced in automated synthetic methods. One or more polynucleotide described herein can also be produced by using an automatic DNA synthesizer. Customized nucleic acids can be ordered from a variety of commercial sources (e.g., ATUM (DNA 2.0), Newark, CA, USA; Life Tech (GeneArt), Carlsbad, CA, USA; GenScript, Ontario, Canada; Base Clear B. V., Leiden, Netherlands; Integrated DNA Technologies, Skokie, IL, USA; Ginkgo Bioworks (Gen9), Boston, MA, USA; and Twist Bioscience, San Francisco, CA, USA). Other techniques for synthesizing nucleic acids and related principles are described by, for example, Itakura et al., Ann. Rev. Biochem. 53:323 (1984) and Itakura et al., Science 198:1056 (1984).

Recombinant DNA techniques useful in modification of nucleic acids are well known in the art, such as, for example, restriction endonuclease digestion, ligation, reverse transcription and cDNA production, and polymerase chain reaction (e.g., PCR). One or more polynucleotide described herein may also be obtained by screening cDNA libraries using one or more oligonucleotide probes that can hybridize to or PCR-amplify polynucleotides which encode one or more variant lipolytic enzyme described herein, or recombinant polypeptide or active fragment thereof. Procedures for screening and isolating cDNA clones and PCR amplification procedures are well known to those of skill in the art and described in standard references known to those skilled in the art. One or more polynucleotide described herein can be obtained by altering a naturally occurring polynucleotide backbone (e.g., that encodes one or more variant lipolytic enzyme described herein or reference lipolytic enzyme) by, for example, a known mutagenesis procedure (e.g., site-directed mutagenesis, site saturation mutagenesis, and in vitro recombination). A variety of methods are known in the art that are suitable for generating modified polynucleotides described herein that encode one or more variant lipolytic enzyme described herein, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches.

A further embodiment is directed to one or more vector comprising one or more variant lipolytic enzyme described herein (e.g., a polynucleotide encoding one or more variant lipolytic enzyme described herein); expression vectors or expression cassettes comprising one or more nucleic acid or polynucleotide sequence described herein; isolated, substantially pure, or recombinant DNA constructs comprising one or more nucleic acid or polynucleotide sequence described herein; isolated or recombinant cells comprising one or more polynucleotide sequence described herein; and compositions comprising one or more such vector, nucleic acid, expression vector, expression cassette, DNA construct, cell, cell culture, or any combination or mixtures thereof.

Some embodiments are directed to one or more recombinant cell comprising one or more vector (e.g., expression vector or DNA construct) described herein which comprises one or more nucleic acid or polynucleotide sequence described herein. Some such recombinant cells are transformed or transfected with such at least one vector, although other methods are available and known in the art. Such cells are typically referred to as host cells. Some such cells comprise bacterial cells, including, but not limited to Bacillus sp. cells, such as B. subtilis cells. Other embodiments are directed to recombinant cells (e.g., recombinant host cells) comprising one or more variant lipolytic enzyme described herein.

In some embodiments, one or more vector described herein is an expression vector or expression cassette comprising one or more polynucleotide sequence described herein operably linked to one or more additional nucleic acid segments required for efficient gene expression (e.g., a promoter operably linked to one or more polynucleotide sequence described herein). A vector may include a transcription terminator and/or a selection gene (e.g., an antibiotic resistant gene) that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pC194, pJH101, pE194, pHP13 (See, Harwood and Cutting [eds.], Chapter 3, Molecular Biological Methods for Bacillus, John Wiley & Sons (1990); suitable replicating plasmids for B. subtilis include those listed on p. 92). (See also, Perego, “Integrational Vectors for Genetic Manipulations in Bacillus subtilis”; Sonenshein et al., [eds.]; “Bacillus subtilis and Other Gram-Positive Bacteria: Biochemistry, Physiology and Molecular Genetics”, American Society for Microbiology, Washington, D.C. (1993), pp. 615-624); and p2JM103BBI).

For expression and production of a protein of interest (e.g., one or more variant lipolytic enzyme described herein) in a cell, one or more expression vector comprising one or more copy of a polynucleotide encoding one or more variant lipolytic enzyme described herein, and in some instances comprising multiple copies, is transformed into the cell under conditions suitable for expression of the variant. In some embodiments, a polynucleotide sequence encoding one or more variant lipolytic enzyme described herein (as well as other sequences included in the vector) is integrated into the genome of the host cell, while in other embodiments, a plasmid vector comprising a polynucleotide sequence encoding one or more variant lipolytic enzyme described herein remains as autonomous extra-chromosomal element within the cell. Some embodiments provide both extrachromosomal nucleic acid elements as well as incoming nucleotide sequences that are integrated into the host cell genome. The vectors described herein are useful for production of the one or more variant lipolytic enzyme described herein. In some embodiments, a polynucleotide construct encoding one or more variant lipolytic enzyme described herein is present on an integrating vector that enables the integration and optionally the amplification of the polynucleotide encoding the variant into the host chromosome. Examples of sites for integration are well known to those skilled in the art. In some embodiments, transcription of a polynucleotide encoding one or more variant lipolytic enzyme described herein is effectuated by a promoter that is the wild-type promoter for the parent enzyme. In some other embodiments, the promoter is heterologous to the one or more variant lipolytic enzyme described herein, but is functional in the host cell. Exemplary promoters for use in bacterial host cells include, but are not limited to the amyE, amyQ, amyL, pstS, sacB, pSPAC, pAprE, pVeg, pHpaII promoters; the promoter of the B. stearothermophilus maltogenic amylase gene; the B. amyloliquefaciens (BAN) amylase gene; the B. subtilis alkaline protease gene; the B. clausii alkaline protease gene; the B. pumilis xylosidase gene; the B. thuringiensis cryIIIA; and the B. licheniformis alpha-amylase gene. Additional promoters include, but are not limited to the A4 promoter, as well as phage Lambda PR or PL promoters and the E. coli lac, trp or tac promoters.

One or more variant lipolytic enzyme described herein can be produced in host cells of any suitable microorganism, including bacteria and fungi. In some embodiments, one or more variant lipolytic enzyme described herein can be produced in Gram-positive bacteria. In some embodiments, the host cells are Bacillus spp., Streptomyces spp., Escherichia spp., Aspergillus spp., Trichoderma spp., Pseudomonas spp., Corynebacterium spp., Saccharomyces spp., or Pichia spp. In some embodiments, one or more variant lipolytic enzyme described herein is produced by Bacillus sp. host cells. Examples of Bacillus sp. host cells that find use in the production of the one or more variant lipolytic enzyme described herein include, but are not limited to B. licheniformis, B. lentus, B. subtilis, B. amyloliquefaciens, B. brevis, B. stearothermophilus, B. alkalophilus, B. coagulans, B. circulans, B. pumilis, B. thuringiensis, B. clausii, and B. megaterium, as well as other organisms within the genus Bacillus. In some embodiments, B. subtilis host cells are used to produce the variants described herein. U.S. Pat. Nos. 5,264,366 and 4,760,025 (RE 34,606) describe various Bacillus host strains that can be used to produce one or more variant lipolytic enzyme described herein, although other suitable strains can be used.

Several bacterial strains that can be used to produce one or more variant lipolytic enzyme described herein include non-recombinant (i.e., wild-type) Bacillus sp. strains, as well as variants of naturally-occurring strains and/or recombinant strains. In some embodiments, the host strain is a recombinant strain, wherein a polynucleotide encoding one or more variant lipolytic enzyme described herein has been introduced into the host. In some embodiments, the host strain is a B. subtilis host strain and particularly a recombinant B. subtilis host strain. Numerous B. subtilis strains are known, including, but not limited to for example, 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211 strain (See e.g., Hoch et al., Genetics 73:215-228 (1973); See also, U.S. Pat. Nos. 4,450,235; 4,302,544; and EP 0134048). The use of B. subtilis as an expression host cell is well known in the art (See e.g., Palva et al., Gene 19:81-87 (1982); Fahnestock and Fischer, J. Bacteriol., 165:796-804 (1986); and Wang et al., Gene 69:39-47 (1988)).

In some embodiments, the Bacillus host cell is a Bacillus sp. that includes a mutation or deletion in at least one of the following genes: degU, degS, degR and degQ. In some embodiments, the mutation is in a degU gene, and in some embodiments the mutation is degU(Hy)32 (See e.g., Msadek et al., J. Bacteriol. 172:824-834 (1990); and Olmos et al., Mol. Gen. Genet. 253:562-567 (1997)). In some embodiments, the Bacillus host comprises a mutation or deletion in scoC4 (See e.g., Caldwell et al., J. Bacteriol. 183:7329-7340 (2001)); spoIIE (See e.g., Arigoni et al., Mol. Microbiol. 31:1407-1415 (1999)); and/or oppA or other genes of the opp operon (See e.g., Perego et al., Mol. Microbiol. 5:173-185 (1991)). Indeed, it is contemplated that any mutation in the opp operon that causes the same phenotype as a mutation in the oppA gene will find use in some embodiments of the altered Bacillus strain described herein. In some embodiments, these mutations occur alone, while in other embodiments, combinations of mutations are present. In some embodiments, an altered Bacillus host cell strain that can be used to produce one or more variant lipolytic enzyme described herein is a Bacillus host strain that already includes a mutation in one or more of the above-mentioned genes. In addition, Bacillus sp. host cells that comprise mutation(s) and/or deletion(s) of endogenous protease genes find use. In some embodiments, the Bacillus host cell comprises a deletion of the aprE and the nprE genes. In other embodiments, the Bacillus sp. host cell comprises a deletion of 5 protease genes, while in other embodiments the Bacillus sp. host cell comprises a deletion of 9 protease genes (See e.g., US 2005/0202535).

Host cells are transformed with one or more nucleic acid sequence encoding one or more variant lipolytic enzyme described herein using any suitable method known in the art. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Exemplary methods for introducing one or more nucleic acid sequence described herein into Bacillus cells are described in, for example, Ferrari et al., “Genetics,” in Harwood et al. [eds.], Bacillus, Plenum Publishing Corp. (1989), pp. 57-72; Saunders et al., J. Bacteriol. 157:718-726 (1984); Hoch et al., J. Bacteriol. 93:1925-1937 (1967); Mann et al., Current Microbiol. 13:131-135 (1986); Holubova, Folia Microbiol. 30:97 (1985); Chang et al., Mol. Gen. Genet. 168:11-115 (1979); Vorobjeva et al., FEMS Microbiol. Lett. 7:261-263 (1980); Smith et al., Appl. Env. Microbiol. 51:634 (1986); Fisher et al., Arch. Microbiol. 139:213-217 (1981); and McDonald, J. Gen. Microbiol. 130:203 (1984)). Indeed, such methods as transformation, including protoplast transformation and transfection, transduction, and protoplast fusion are well known and suited for use herein. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 (1979); Haima et al., Mol. Gen. Genet. 223:185-191 (1990); Weinrauch et al., J. Bacteriol. 154:1077-1087 (1983); and Weinrauch et al., J. Bacteriol. 169:1205-1211 (1987)). In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising a nucleic acid encoding one or more variant lipolytic enzyme described herein (i.e., an intermediate cell is not used to amplify, or otherwise process, the DNA construct or vector prior to introduction into the host cell). Introduction of a DNA construct or vector described herein into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, and liposomes. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 (1984); and Palmeros et al., Gene 247:255-264 (2000)).

In some embodiments, the transformed cells are cultured in conventional nutrient media. The suitable specific culture conditions, such as temperature, pH and the like are known to those skilled in the art and are well described in the scientific literature. Some embodiments provide a culture (e.g., cell culture) comprising one or more variant lipolytic enzyme or nucleic acid sequence described herein.

In some embodiments, host cells transformed with one or more polynucleotide sequence encoding one or more variant lipolytic enzyme described herein are cultured in a suitable nutrient medium under conditions permitting the expression of the variant, after which the resulting variant is recovered from the culture. In some embodiments, the variant produced by the cells is recovered from the culture medium by conventional procedures, including, but not limited to, for example, separating the host cells from the medium by centrifugation or filtration, precipitating the proteinaceous components of the supernatant or filtrate by means of a salt (e.g., ammonium sulfate), and chromatographic purification (e.g., ion exchange, gel filtration, affinity, etc.).

In some embodiments, one or more variant lipolytic enzyme produced by a recombinant host cell is secreted into the culture medium. A nucleic acid sequence that encodes a purification facilitating domain may be used to facilitate purification of the variant. A vector or DNA construct comprising a polynucleotide sequence encoding one or more variant lipolytic enzyme described herein may further comprise a nucleic acid sequence encoding a purification facilitating domain to facilitate purification of the variant (See e.g., Kroll et al., DNA Cell Biol. 12:441-53 (1993)). Such purification facilitating domains include, but are not limited to, for example, metal chelating peptides such as histidine-tryptophan modules that allow purification on immobilized metals (See, Porath, Protein Expr. Purif. 3:263-281 [1992]), protein A domains that allow purification on immobilized immunoglobulin, and the domain utilized in the FLAGS extension/affinity purification system. The inclusion of a cleavable linker sequence such as Factor XA or enterokinase (e.g., sequences available from Invitrogen, San Diego, CA) between the purification domain and the heterologous protein also find use to facilitate purification.

The present variant proteins can be produced in host cells, for example, by secretion or intracellular expression, using methods well-known in the art. Fermentation, separation, and concentration techniques are well known in the art and conventional methods can be used to prepare a concentrated, enzyme-containing solution. Host cells may be further processed, such as to release enzyme or to improve cell separation, for example by heating or by changing pH or salt content or by treating with enzymes including hen egg white lysozyme, T4 lysozyme, or enzymes described in WO2022047149_For production scale recovery, variant polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

A variety of methods can be used to determine the level of production of one or more mature variant lipolytic enzyme described herein in a host cell. Such methods include, but are not limited to, for example, methods that utilize either polyclonal or monoclonal antibodies specific for the enzyme. Exemplary methods include, but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox et al., J. Exp. Med. 158:1211 (1983)). In another embodiment, the method that can be used includes the assays provided in Examples 2 and 3.

Some other embodiments provide methods for making or producing one or more mature variant lipolytic enzyme described herein. A mature variant does not include a signal peptide or a propeptide sequence. Some methods comprise making or producing one or more variant lipolytic enzyme described herein in a recombinant bacterial host cell, such as for example, a Bacillus sp. cell (e.g., a B. subtilis cell). Other embodiments provide a method of producing one or more variant described herein, wherein the method comprises cultivating a recombinant host cell comprising a recombinant expression vector comprising a nucleic acid sequence encoding one or more variant lipolytic enzyme described herein under conditions conducive to the production of the variant. Some such methods further comprise recovering the variant from the culture.

Further embodiments provide methods of producing one or more variant lipolytic enzyme described herein, wherein the methods comprise: (a) introducing a recombinant expression vector comprising a nucleic acid encoding the variant into a population of cells (e.g., bacterial cells, such as B. subtilis cells); and (b) culturing the cells in a culture medium under conditions conducive to produce the variant encoded by the expression vector. Some such methods further comprise: (c) isolating the variant from the cells or from the culture medium.

### Compositions

The disclosure further provides compositions (e.g. cleaning or detergent compositions) comprising a variant lipolytic enzyme as provided herein. The compositions generally comprise a variant lipolytic enzyme as provided herein and one or more additional detergent components, such as a surfactant.

Such compositions include detergent or cleaning compositions. As used herein, the term “detergent composition” or “detergent formulation” is used in reference to a composition intended for use in a wash medium (e.g. a wash liquor) for the cleaning or treatment of soiled or dirty objects, including particular textile or non-textile objects or items. Such compositions of the present invention are not limited to any particular detergent composition or formulation. Indeed, in some embodiments, the detergents of the invention comprise at least one variant lipolytic enzyme as provided herein and, in addition, one or more surfactants, transferase(s), additional hydrolytic enzymes, oxido reductases, builders (e.g., a builder salt), bleaching agents, bleach activators, bluing agents, fluorescent dyes, caking inhibitors, masking agents, enzyme activators, antioxidants, and/or solubilizers. In some instances, a builder salt is a mixture of a silicate salt and a phosphate salt, preferably with more silicate (e.g., sodium metasilicate) than phosphate (e.g., sodium tripolyphosphate). Some compositions of the invention, such as, but not limited to, cleaning compositions or detergent compositions, do not contain any phosphate (e.g., phosphate salt or phosphate builder).

The compositions having a variant lipolytic enzyme, which find use in the methods provided herein, may comprise a variant lipolytic enzyme at a concentration of in use of 0.001 to 10,000 mg/L, or 0.001 to 2000 mg/L, or 0.01 to 5000 mg/L, or 0.01 to 2000 mg/L, or 0.01 to 1300 mg/L, or 0.1 to 5000 mg/L, or 0.1 to 2000 mg/L, or 0.1 to 1300 mg/L, or 1 to 5000 mg/L, or 1 to 1300 mg/L, or 1 to 500 mg/L, or 10 to 5000 mg/L, or 10 to 1300 mg/L, or 10 to 500 mg/L. In another embodiment, the composition may contain a variant lipolytic enzyme in an amount of 0.002 to 5000 mg of protein, such as 0.005 to 1300 mg of protein, or 0.01 to 5000 mg of protein, or 0.01 to 1300 mg of protein, or 0.1 to 5000 mg of protein, or 1 to 1300 mg of protein, preferably 0.1 to 1300 mg of protein, more preferably 1 to 1300 mg of protein, even more preferably 10 to 500 mg of protein, per liter of wash liquor, or in the amount of at least 0.01 ppm active lipase.

In one embodiment, the composition comprises a variant lipolytic enzyme as provided herein and at least one additional detergent component, and optionally one or more additional enzymes.

In some embodiments, the cleaning or detergent compositions of the present invention further comprise adjunct materials including, but not limited to, surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (See e.g., U.S. Pat. Nos. 6,610,642, 6,605,458, 5,705,464, 5,710,115, 5,698,504, 5,695,679, 5,686,014 and 5,646,101, all of which are incorporated herein by reference).

The detergent or cleaning compositions of the present disclosure are advantageously employed for example, in laundry applications, hard surface cleaning, dishwashing applications, as well as cosmetic applications such as dentures, teeth, hair and skin. In addition, in some embodiments, the variant lipolytic enzyme of the present invention are ideally suited for laundry applications. Furthermore, the variants of the present disclosure find use in granular and liquid compositions.

Enzyme component weights are based on total active protein. All percentages and ratios are calculated by weight unless otherwise indicated. All percentages and ratios are calculated based on the total composition unless otherwise indicated. In laundry detergent compositions, the enzyme levels are expressed in ppm, which equals mg active protein/kg detergent composition.

In some embodiments, the laundry detergent compositions described herein further comprise a surfactant. In some embodiments, the surfactant is selected from a non-ionic, ampholytic, semi-polar, anionic, cationic, zwitterionic, and combinations and mixtures thereof. In yet a further embodiment, the surfactant is selected from an anionic surfactant, a cationic surfactant, a zwitterionic surfactant, and combinations thereof. In some embodiments, the laundry detergent compositions described herein comprise from about 0.1% to about 60%, about 1% to about 50%, or about 5% to about 40% surfactant by weight of the composition.

Exemplary surfactants include, but are not limited to sodium dodecylbenzene sulfonate, C12-14 pareth-7, C12-15 pareth-7, sodium C12-15 pareth sulfate, C14-15 pareth-4, sodium laureth sulfate (e.g., Steol CS-370), sodium hydrogenated cocoate, C12 ethoxylates (Alfonic 1012-6, Hetoxol LA7, Hetoxol LA4), sodium alkyl benzene sulfonates (e.g., Nacconol 90G), and combinations and mixtures thereof. Anionic surfactants include but are not limited to linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. Nonionic surfactants include but are not limited to alcohol ethoxylate (AEO or AE), carboxylated alcohol ethoxylates, nonylphenol ethoxylate, alkylpolyglycoside, alkyldimethylamine oxide, ethoxylated fatty acid monoethanolamide, fatty acid monoethanolamide, polyhydroxy alkyl fatty acid amide (e.g., as described in WO92/06154), polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p-tert-octylphenols or octylphenyl-ethylene oxide condensates (e.g., NONIDET P40), ethylene oxide condensates with fatty alcohols (e.g., LUBROL), polyoxyethylene nonylphenols, polyalkylene glycols (SYNPERONIC F108), sugar-based surfactants (e.g., glycopyranosides, thioglycopyranosides), and combinations and mixtures thereof.

In a further embodiment, the laundry detergent compositions described herein further comprise a surfactant mixture that includes, but is not limited to 5-15% anionic surfactants, <5% nonionic surfactants, cationic surfactants, phosphonates, soap, enzymes, perfume, butylphenyl methylpropionate, geraniol, zeolite, polycarboxylates, hexyl cinnamal, limonene, cationic surfactants, citronellol, and benzisothiazolinone.

The laundry detergent compositions described herein may additionally include one or more detergent builders or builder systems, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, an optical brightener, a fabric conditioner, and a perfume. The laundry detergent compositions described herein may also include additional enzymes selected from proteases, amylases, cellulases, lipases, mannanases, nucleases, pectinases, xyloglucanases, or perhydrolases, as provided in more detail herein.

In some embodiments, the laundry detergent compositions described herein further comprises from about 1%, from about 3% to about 60% or even from about 5% to about 40% builder by weight of the cleaning composition. Builders may include, but are not limited to, the alkali metals, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metals, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). Any suitable builder can find use in the compositions described herein, including those known in the art.

In some embodiments, the laundry detergent compositions described herein further comprise an adjunct ingredient including, but not limited to surfactants, builders, bleaches, bleach activators, bleach catalysts, additional enzymes, an enzyme stabilizer (including, for example, an enzyme stabilizing system), chelants, optical brighteners, soil release polymers, dye transfer agents, dye transfer inhibiting agents, catalytic materials, hydrogen peroxide, sources of hydrogen peroxide, preformed peracids, polymeric dispersing agents, clay soil removal agents, structure elasticizing agents, dispersants, suds suppressors, dyes, perfumes, colorants, filler salts, hydrotropes, photoactivators, fluorescers, fabric conditioners, hydrolyzable surfactants, solvents, preservatives, anti-oxidants, anti-shrinkage agents, anti-wrinkle agents, germicides, fungicides, color speckles, anti-corrosion agents, alkalinity sources, solubilizing agents, carriers, processing aids, pigments, pH control agents, and combinations thereof. (See, e.g., U.S. Pat. Nos. 6,610,642, 6,605,458, 5,705,464, 5,710,115, 5,698,504, 5,695,679, 5,686,014, and 5,646,101). In some embodiments, one or more adjunct is incorporated for example, to assist or enhance cleaning performance, for treatment of the substrate to be cleaned, or to modify the aesthetics of the cleaning composition as is the case with perfumes, colorants, dyes or the like. Any such adjunct ingredient is in addition to variant enzyme provided herein. In some embodiments, the adjunct ingredient is selected from surfactants, enzyme stabilizers, builder compounds, polymeric compounds, bleaching agents, additional enzymes, suds suppressors, dispersants, lime-soap dispersants, soil suspension agents, softening agents, anti-redeposition agents, corrosion inhibitors, and combinations thereof.

In some further embodiments, the laundry detergent compositions described herein comprise one or more enzyme stabilizer. In some embodiments, the enzyme stabilizer is a water-soluble source of calcium and/or magnesium ions. In some embodiments, the enzyme stabilizers include oligosaccharides, polysaccharides, and inorganic divalent metal salts, including alkaline earth metals, such as calcium salts. In some embodiments, the enzymes employed herein are stabilized by the presence of water-soluble sources of zinc (II), calcium (H) and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV)). Chlorides and sulfates also find use in some embodiments. Exemplary oligosaccharides and polysaccharides (e.g., dextrins) are described, for example, in WO07145964. In some embodiments, the laundry detergent compositions described herein contain reversible protease inhibitors selected from a boron-containing compound (e.g., borate, 4-formyl phenyl boronic acid, and phenyl-boronic acid derivatives, such as, e.g., are described in WO9641859); a peptide aldehyde (such as, e.g., is described in WO2009118375 and WO2013004636), and combinations thereof.

The cleaning compositions herein are typically formulated such that, during use in aqueous cleaning operations, the wash water will have a pH of from about 3.0 to about 11. Liquid product formulations are typically formulated to have a neat pH from about 5.0 to about 9.0, more preferably from about 7.5 to about 9. Granular laundry products are typically formulated to have a pH from about 8.0 to about 11.0. Techniques for controlling pH at recommended usage levels include the use of buffers, alkalis, acids, etc., and are well known to those skilled in the art.

Suitable high pH cleaning compositions typically have a neat pH of from about 9.0 to about 11.0, or even a neat pH of from 9.5 to 10.5. Such cleaning compositions typically comprise a sufficient amount of a pH modifier, such as sodium hydroxide, monoethanolamine, or hydrochloric acid, to provide such cleaning composition with a neat pH of from about 9.0 to about 11.0. Such compositions typically comprise at least one base-stable enzyme. In some embodiments, the compositions are liquids, while in other embodiments, they are solids.

In one embodiment, the cleaning compositions include those having a pH of from 7.4 to pH 11.5, or pH 7.4 to pH 11.0, or pH 7.5 to pH 11.5, or pH 7.5 to pH 11.0, or pH 7.5 to pH 10.5, or pH 7.5 to pH 10.0, or pH 7.5 to pH 9.5, or pH 7.5 to pH 9.0, or pH 7.5 to pH 8.5, or pH 7.5 to pH 8.0, or pH 7.6 to pH 11.5, or pH 7.6 to pH 11.0, or pH 7.6 to pH 10.5, or pH 8.7 to pH 10.0, or pH 8.0 to pH 11.5, or pH 8.0 to pH 11.0, or pH 8.0 to pH 10.5, or pH 8.0 to pH 10.0.

Concentrations of detergent compositions in typical wash solutions throughout the world vary from less than about 800 ppm of detergent composition (“low detergent concentration geographies”), for example about 667 ppm in Japan, to between about 800 ppm to about 2000 ppm (“medium detergent concentration geographies”), for example about 975 ppm in U.S. and about 1500 ppm in Brazil, to greater than about 2000 ppm (“high detergent concentration geographies”), for example about 4500 ppm to about 5000 ppm in Europe and about 6000 ppm in high suds phosphate builder geographies.

In some embodiments, the detergent compositions described herein may be utilized at a temperature of from about 10° C. to about 60° C., or from about 20° C. to about 60° C., or from about 30° C. to about 60° C., from about 40° C. to about 60° C., from about 40° C. to about 55° C., or all ranges within 10° C. to 60° C. In some embodiments, the detergent compositions described herein are used in “cold water washing” at temperatures of from about 10° C. to about 40° C., or from about 20° C. to about 30° C., from about 15° C. to about 25° C., from about 15° C. to about 35° C., or all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm #divided by 17.1 equals grains per gallon) of hardness minerals.

TABLE I
Water Hardness Levels

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

In other embodiments, the composition described herein comprises one or more additional enzyme. The one or more additional enzyme is selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, DNases, endo-beta-1, 4-glucanases, endo-beta-mannanases, esterases, exo-mannanases, galactanases, glucoamylases, hemicellulases, hexoaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, peroxidases, phenoloxidases, phosphatases, phospholipases, phytases, polygalacturonases, polyesterases, additional proteases, pullulanases, reductases, rhamnogalacturonases, beta-glucanases, tannases, transglutaminases, xylan acetyl-esterases, xylanases, xyloglucanases, xylosidases, and any combination or mixture thereof. Some embodiments are directed to a combination of enzymes (i.e., a “cocktail”) comprising enzymes like amylase, protease, lipase, mannanase, and/or nuclease in conjunction with one or more variant lipolytic enzyme in the compositions provided herein.

In some embodiments, the compositions provided herein comprise a variant lipolytic enzyme in combination with a protease. The protease for use in combination with the variant lipolytic enzyme in the compositions of the instant disclosure include any polypeptide having protease activity. In one embodiment, the additional protease is a serine protease. In another embodiment, the additional protease is a metalloprotease, a fungal subtilisin, or an alkaline microbial protease or a trypsin-like protease. Suitable proteases include those of animal, vegetable or microbial origin. In some embodiments, the protease is a microbial protease. In other embodiments, the protease is a chemically or genetically modified mutant. In another embodiment, the protease is subtilisin like protease or a trypsin-like protease. In other embodiments, the additional protease does not contain cross-reactive epitopes with the variant as measured by antibody binding or other assays available in the art. Exemplary subtilisin proteases include those derived from for example, Bacillus (e.g., BPN′, Carlsberg, subtilisin 309, subtilisin 147, and subtilisin 168), or fungal origin, such as, for example, those described in U.S. Pat. No. 8,362,222. Exemplary additional proteases include but are not limited to those described in WO92/21760, WO95/23221, WO2008/010925, WO09/149200, WO09/149144, WO09/149145, WO 10/056640, WO10/056653, WO2010/0566356, WO 11/072099, WO2011/13022, WO11/140364, WO 12/151534, WO2015/038792, WO2015/089447, WO2015/089441, WO 2017/215925, US Publ. No. 2008/0090747, U.S. Pat. Nos. 5,801,039, 5,340,735, 5,500,364, 5,855,625, RE 34,606, 5,955,340, 5,700,676 6,312,936, 6,482,628, 8,530,219, U.S. Provisional Appl Nos. 62/180,673 and 62/161,077, and PCT Appl Nos. PCT/US2015/021813, PCT/US2015/055900, PCT/US2015/057497, PCT/US2015/057492, PCT/US2015/057512, PCT/US2015/057526, PCT/US2015/057520, PCT/US2015/057502, PCT/US2016/022282, and PCT/US16/32514, International publications WO2016001449, WO2016087617, WO2016096714, WO2016203064, WO2017089093, and WO2019180111, as well as metalloproteases described in WO1999014341, WO1999033960, WO1999014342, WO1999034003, WO2007044993, WO2009058303, WO 2009058661, WO2014071410, WO2014194032, WO2014194034, WO 2014194054, and WO 2014/194117. Exemplary additional proteases include, but are not limited to trypsin (e.g., of porcine or bovine origin) and the Fusarium protease described in WO89/06270. Exemplary commercial proteases include, but are not limited to MAXATASE®, MAXACAL®, MAXAPEM™, OPTICLEAN®, OPTIMASE®, PROPERASE®, PURAFECT®, PURAFECT® OXP, PURAMAX™, EXCELLASE™, PREFERENZ™ proteases (e.g. P100, P110, P280), EFFECTENZ™ proteases (e.g. P1000, P1050, P2000), EXCELLENZ™ proteases (e.g. P1000), ULTIMASE®, and PURAFAST™ (DuPont); ALCALASE®, BLAZE®, BLAZE® variants, BLAZE® EVITY®, BLAZE® EVITY® 16L, CORONASE®, SAVINASE®, SAVINASE® ULTRA, SAVINASE® EVITY®, SAVINASE® EVERIS®, PRIMASE®, DURAZYM™, POLARZYME®, OVOZYME®, KANNASE®, LIQUANASE®, LIQUANASE EVERIS®, NEUTRASE®, PROGRESS UNO®, RELASE®, and ESPERASE® (Novozymes); BLAP™ and BLAP™ variants (Henkel); LAVERGY™ PRO 104 L, LAVERGY™ PRO 106 LS, LAVERGY™ PRO 114 LS (BASF), KAP (B. alkalophilus subtilisin (Kao)) and BIOTOUCH® (AB Enzymes).

In some embodiments, the compositions provided herein comprise a variant lipolytic enzyme in combination with one or more amylases. In one embodiment, the composition comprises from about 0.00001% to about 10%, about 0.0001% to about 10%, about 0.001% to about 5%, about 0.001% to about 2%, or about 0.005% to about 0.5% amylase by weight composition. Any amylase (e.g., alpha and/or beta) suitable for use in alkaline solutions may be useful to include in such composition. An exemplary amylase can be a chemically or genetically modified mutant. Exemplary amylases include, but are not limited to those of bacterial or fungal origin, such as, for example, amylases described in GB 1,296,839, WO9100353, WO9402597, WO94183314, WO9510603, WO9526397, WO9535382, WO9605295, WO9623873, WO9623874, WO 9630481, WO9710342, WO9741213, WO9743424, WO9813481, WO 9826078, WO9902702, WO 9909183, WO9919467, WO9923211, WO9929876, WO9942567, WO 9943793, WO9943794, WO 9946399, WO0029560, WO0060058, WO0060059, WO0060060, WO 0114532, WO0134784, WO 0164852, WO0166712, WO0188107, WO0196537, WO02092797, WO 0210355, WO0231124, WO 2004055178, WO2004113551, WO2005001064, WO2005003311, WO 2005018336, WO2005019443, WO2005066338, WO2006002643, WO2006012899, WO2006012902, WO2006031554, WO 2006063594, WO2006066594, WO2006066596, WO2006136161, WO 2008000825, WO2008088493, WO2008092919, WO2008101894, WO2008/112459, WO2009061380, WO2009061381, WO 2009100102, WO2009140504, WO2009149419, WO 2010/059413, WO 2010088447, WO2010091221, WO2010104675, WO2010115021, WO10115028, WO2010117511, WO 2011076123, WO2011076897, WO2011080352, WO2011080353, WO 2011080354, WO2011082425, WO2011082429, WO 2011087836, WO2011098531, WO2013063460, WO2013184577, WO 2014099523, WO2014164777, and WO2015077126. Exemplary commercial amylases include, but are not limited to AMPLIFY®, DURAMYL®, TERMAMYL®, FUNGAMYL®, STAINZYME®, STAINZYME PLUS®, STAINZYME PLUS®, STAINZYME ULTRA® EVITY®, and BAN™ (Novozymes); EFFECTENZ™ S 1000, POWERASE™, PREFERENZ™ S 100, PREFERENZ™ S 110, EXCELLENZ™ S 2000, RAPIDASE® and MAXAMYL® P (DuPont).

In some embodiments, the compositions provided herein comprise a variant lipolytic enzyme in combination with one or more additional lipases. In some embodiments, the composition comprises from about 0.00001% to about 10%, about 0.0001% to about 10%, about 0.001% to about 5%, about 0.001% to about 2%, or about 0.005% to about 0.5% lipase by weight composition. An exemplary lipase can be a chemically or genetically modified mutant. Exemplary lipases include, but are not limited to, e.g., those of bacterial or fungal origin, such as, e.g., H. lanuginosa lipase (see, e.g., EP 258068 and EP 305216), T. lanuginosa lipase (see, e.g., WO 2014/059360 and WO2015/010009), Rhizomucor miehei lipase (see, e.g., EP 238023), Candida lipase, such as C. antarctica lipase (e.g., C. antarctica lipase A or B) (see, e.g., EP 214761), Pseudomonas lipases such as P. alcaligenes and P. pseudoalcaligenes lipase (see, e.g., EP 218272), P. cepacia lipase (see, e.g., EP 331376), P. stutzeri lipase (see, e.g., GB 1,372,034), P. fluorescens lipase, Bacillus lipase (e.g., B. subtilis lipase (Dartois et al., Biochem. Biophys. Acta 1131:253-260 (1993)), B. stearothermophilus lipase (see, e.g., JP 64/744992), and B. pumilus lipase (see, e.g., WO 91/16422)). Exemplary cloned lipases include, but are not limited to Penicillium camembertii lipase (See, Yamaguchi et al., Gene 103:61-67 (1991)), Geotrichum candidum lipase (See, Schimada et al., J. Biochem., 106:383-388 (1989)), and various Rhizopus lipases, such as, R. delemar lipase (See, Hass et al., Gene 109:117-113 (1991)), R. niveus lipase (Kugimiya et al., Biosci. Biotech. Biochem. 56:716-719 (1992)) and R. oryzae lipase. Other lipolytic enzymes, such as cutinases, may also find use in one or more composition described herein, including, but not limited to, e.g., cutinase derived from Pseudomonas mendocina (see, WO 88/09367) and/or Fusarium solanipisi (see, WO90/09446). Exemplary commercial lipases include, but are not limited to M1 LIPASE™, LUMA FAST™, and LIPOMAX™ (DuPont); LIPEX®, LIPOCLEAN®, LIPOLASE® and LIPOLASE® ULTRA (Novozymes); and LIPASE P™ (Amano Pharmaceutical Co. Ltd).

In some embodiments, the compositions provided herein comprise a variant lipolytic enzyme in combination with one or more mannanases. In one embodiment, the composition comprises from about 0.00001% to about 10%, about 0.0001% to about 10%, about 0.001% to about 5%, about 0.001% to about 2%, or about 0.005% to about 0.5% mannanase by weight composition. An exemplary mannanase can be a chemically or genetically modified mutant. Exemplary mannanases include, but are not limited to, those of bacterial or fungal origin, such as, for example, those described in WO 2016/007929; U.S. Pat. Nos. 6,566,114; 6,602,842; and 6,440,991: and U.S. Provisional Appl. Nos. 62/251,516, 62/278,383, and 62/278,387. Exemplary commercial mannanases include, but are not limited to MANNAWAY® (Novozymes) and EFFECTENZ™ M 1000, EFFECTENZ™ M 2000, PREFERENZ® M 100, MANNASTAR®, and PURABRITE™ (DuPont).

In some embodiments, the compositions and methods provided herein comprise variant lipolytic enzyme in combination with a nuclease, such as a DNase or RNase. Exemplary nucleases include, but are not limited to, those described in WO2015181287, WO2015155350, WO2016162556, WO2017162836, WO2017060475 (e.g. SEQ ID NO: 21), WO2018184816, WO2018177936, WO2018177938, WO2018/185269, WO2018185285, WO2018177203, WO2018184817, WO2019084349, WO2019084350, WO2019081721, WO2018076800, WO2018185267, WO2018185280, and WO2018206553. Other nucleases which can be used in combination with the variant lipolytic enzymes in the compositions and methods provided herein include those described in Nijland R, Hall M J, Burgess J G (2010) Dispersal of Biofilms by Secreted, Matrix Degrading, Bacterial DNase. PLoS ONE 5(12) and Whitchurch, C. B., Tolker-Nielsen, T., Ragas, P. C., Mattick, J. S. (2002) Extracellular DNA required for bacterial biofilm formation. Science 295: 1487.

Yet a still further embodiment is directed to a composition comprising one or more variant lipolytic enzymes described herein and one or more cellulase. In one embodiment, the composition comprises from about 0.00001% to about 10%, 0.0001% to about 10%, about 0.001% to about 5%, about 0.001% to about 2%, or about 0.005% to about 0.5% cellulase by weight of composition. Any suitable cellulase may find use in a composition described herein. An exemplary cellulase can be a chemically or genetically modified mutant. Exemplary cellulases include but are not limited, to those of bacterial or fungal origin, such as, for example, those described in WO2005054475, WO2005056787, U.S. Pat. Nos. 7,449,318, 7,833,773, 4,435,307; EP 0495257; and U.S. Provisional Appl. No. 62/296,678. Exemplary commercial cellulases include, but are not limited to, CELLUCLEAN®, CELLUZYME®, CAREZYME®, ENDOLASE®, RENOZYME®, and CAREZYME® PREMIUM (Novozymes); REVITALENZ™ 100, REVITALENZ™ 200/220, and REVITALENZ® 2000 (DuPont); and KAC-500(B)™ (Kao Corporation). In some embodiments, cellulases are incorporated as portions or fragments of mature wild-type or variant cellulases, wherein a portion of the N-terminus is deleted (see, e.g., U.S. Pat. No. 5,874,276).

In some embodiments, the laundry detergent compositions described herein comprise at least one chelating agent. Suitable chelating agents may include, but are not limited to copper, iron, and/or manganese chelating agents, and mixtures thereof. In some embodiments, the laundry detergent compositions described herein comprises from about 0.1% to about 15% or even from about 3.0% to about 10% chelating agent by weight of composition.

In some still further embodiments, the laundry detergent compositions described herein comprise at least one deposition aid. Suitable deposition aids include, but are not limited to, polyethylene glycol, polypropylene glycol, polycarboxylate, soil release polymers such as polyterephthalic acid, clays such as kaolinite, montmorillonite, attapulgite, illite, bentonite, halloysite, and mixtures thereof.

In some embodiments, the laundry detergent compositions described herein comprise at least one anti-redeposition agent.

In some embodiments, the laundry detergent compositions described herein comprise one or more dye transfer inhibiting agent. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, and polyvinylimidazoles, or mixtures thereof. In some embodiments, the laundry detergent compositions described herein comprise from about 0.0001% to about 10%, from about 0.01% to about 5%, or even from about 0.1% to about 3% dye transfer inhibiting agent by weight of composition.

In some embodiments, the laundry detergent compositions described herein comprise one or more silicates. In some such embodiments, sodium silicates (e.g., sodium disilicate, sodium metasilicate, and crystalline phyllosilicates) find use. In some embodiments, the laundry detergent compositions described herein comprise from about 1% to about 20% or from about 5% to about 15% silicate by weight of the composition.

In yet further embodiments, the laundry detergent compositions described herein comprise one or more dispersant. Suitable water-soluble organic materials include, but are not limited to the homo- or co-polymeric acids or their salts, in which the polycarboxylic acid comprises at least two carboxyl radicals separated from each other by not more than two carbon atoms.

In some embodiments, the laundry detergent compositions described herein comprise one or more bleach, bleach activator, and/or bleach catalyst. In some embodiments, the laundry detergent compositions described herein comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Suitable salts include, for example, those described in EP2100949. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxycarboxylic acids having preferably from about 1 to about 10 carbon atoms, in particular from about 2 to about 4 carbon atoms, and/or optionally substituted perbenzoic acid. Bleach catalysts typically include, for example, manganese triazacyclononane and related complexes, and cobalt, copper, manganese, and iron complexes, as well as those described in U.S. Pat. Nos. 4,246,612, 5,227,084, 4,810,410, WO9906521, and EP2100949.

In some embodiments, the laundry detergent compositions described herein comprise one or more catalytic metal complex. In some embodiments, a metal-containing bleach catalyst finds use. In other embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See, e.g., U.S. Pat. No. 4,430,243). In some embodiments, the laundry detergent compositions described herein are catalyzed by means of a manganese compound. Such compounds and levels of use are well known in the art (See, e.g., U.S. Pat. No. 5,576,282). In additional embodiments, cobalt bleach catalysts find use in the laundry detergent compositions described herein. Various cobalt bleach catalysts are known in the art (See, e.g., U.S. Pat. Nos. 5,597,936 and 5,595,967) and are readily prepared by known procedures.

Polyesters as used herein include polymers that contain at least one ester repeating unit in their main chain polymers. In their simplest form, polyesters are produced by polycondensation reaction of a glycol (diol) with a dicarboxylic acid (diacid) or its diester. Polyesters include naturally occurring chemicals, such as in the cutin of plant cuticles, as well as synthetics through step-growth polymerization such as polybutyrate.

Polyesters that can be contacted with the variant lipases provided herein (e.g. in the methods provided herein), or a composition including such variant lipase include any ester bond-containing polymer. Such polyesters include aliphatic and aromatic polyesters. The aliphatic polyesters include: polyhydroxyalkanoates (PHA), which can be divided into polyhydroxybutyrate (PHB), polyhydroxyvalerate (PHV), polyhydroxyhexanoate (PHH), and their copolymers; polylactide (PLA); poly(ε-caprolactone) (PCL); polybutylenesuccinate (PBS) and its derivative poly(butylenesuccinate adipate) (PBSA). The aromatic polyesters include: modified poly(ethylene terephthalate) (PET) such as poly(butylene adipate/terephthalate) (PBAT) and poly(tetramethylene adipate-coterephthalate) (PTMAT); and aliphatic-aromatic copolyesters (AAC). In some embodiments, polyesters may be partially or substantially biodegradable. In other embodiments, the polyesters may be partially or substantially resistant to microbial and enzymatic attack.

In some embodiments, a polyester may be an aliphatic polyester. In some embodiments, a polyester may be an aromatic polyester. In some embodiments, an aromatic polyester maybe a polyethylene terephthalate (PET). In some embodiments, an aromatic polyester maybe a polytrimethylene terephthalate (PTT).

Accordingly, in one embodiment, the polyesters that find use in the methods provided herein include those selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

In another embodiment, the fabrics or textiles that find use in the methods provided herein include fabrics and textiles that contain at least one polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

In some embodiments, the disclosure provides methods for treating a fabric or a textile comprising contacting a fabric or a textile with a variant lipolytic enzyme as provided herein, or a composition comprising such variant lipolytic enzyme and optionally rinsing the fabric or textile.

In some embodiments, the contacting steps of the methods provided herein comprise a variant lipolytic enzyme in an amount selected from the group consisting of 0.002 to 10,000 mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor.

### Esterase for Surface Modification

In some embodiments, a polyester (e.g. PET)-containing textile, fabric, or film may have a hydrolyzable polymer end or a loop on their surface. The variant lipolytic enzymes provided herein find use in surface modification of polyester (e.g. PET) fibers, which may improve factors such as finishing fastness, dyeability, wettability, and de-pilling. In some embodiments, polymer chains that protrude or form a loop on the surface of a polyester (e.g. PET)-containing textile, fiber or film may be hydrolyzed by the variant lipases herein to carboxylic acid and hydroxyl residues, thus increasing surface hydrophilicity. Pilling is the formation of small, fuzzy balls on the surface of polyester (e.g. PET) fabrics resulting in an unsightly worn appearance of the textile. Generally, these nodules are produced by loose fibers in the fabric or those which have been released from the tissue.

Thus, in some embodiments, the variant lipolytic enzymes of the present disclosure can be used for finishing fastness, dyeability, wettability, and de-pilling of polyester (e.g. PET) textiles, fabrics, and films. In other embodiments, the variant lipolytic enzymes of the present disclosure may be used in a detergent composition in order to reduce pilling during textile cleaning. In some embodiments, the variant lipolytic enzymes of the present disclosure have PETase activity.

In one embodiment, methods for degrading a polyester or a polyester-containing material are provided, where the methods comprise contacting a polyester-containing material with a variant lipolytic enzyme or composition comprising a variant lipolytic enzyme as provided herein. In some embodiments, the polyester-containing material is a polyester textile or fabric.

In another embodiment, the disclosure provides a method for the enzymatic depolymerization of a polyester-containing material, where the method comprises contacting a polyester-containing material with a variant lipolytic enzyme or composition comprising a variant lipolytic enzyme as provided herein, and recovering monomers and/or oligomers of the polyester. In some embodiments, the polyester-containing material is a polyester textile or fabric.

The textile or fabric can be contacted with the variant lipolytic enzyme or a composition comprising the variant lipolytic enzyme in a washing machine or in a manual wash tub (e.g. for handwashing). In one embodiment, the textile or fabric is contacted with the variant lipolytic enzyme or a composition comprising the variant lipolytic enzyme in a wash liquor. In another embodiment, a solution containing the variant lipolytic enzyme is incubated with or flowed over the polyester-containing material, such as by pumping the solution through tubing or pipes or by filling a reservoir with the solution.

In some embodiments, the textiles or articles are contacted with the variant lipolytic enzyme or a composition comprising the variant lipolytic enzyme under conditions having a temperature that allows for activity of the variant lipolytic enzyme. In some embodiments, the temperature in the methods disclosed herein include those between 10° to 60° C., between 10° to about 45° C., between 15° to about 55° C., between 15° to about 50° C., between 15° to about 45° C., between 20° to about 60° C., between 20° to about 50° C. and between 20° to about 45° C.

The polypeptides, compositions, and methods provided herein have utility in a wide array of applications in which degrading polyester (e.g. PET) is desired, such as household cleaning, including in washing machines, dishwashers, and on household surfaces.

Other aspects and embodiments of the present compositions and methods will be apparent from the foregoing description and following examples. Various alternative embodiments beyond those described herein can be employed in practicing the invention without departing from the spirit and scope of the invention. Accordingly, the claims, and not the specific embodiments described herein, define the scope of the invention and as such methods and structures within the scope of the claims and their equivalents are covered thereby.

### EMBODIMENTS

Embodiment 1. A variant lipolytic enzyme comprising an amino acid sequence having at least 70% identity to the full length amino acid sequence of SEQ ID NO: 2, comprising one or more substitutions at positions selected from the group consisting of 14, 70, 117, 161, 175, 212, 226, 236, 239, 252, 254, 256, and 258, wherein the positions are numbered by reference to the amino acid sequence of SEQ ID NO: 2, and wherein the variant has esterase activity.

Embodiment 2. The variant lipolytic enzyme of embodiment 1, wherein the variant comprises at least one additional mutation at a position selected from the group consisting of 40, 59, 61, 64, 66, 177, 178, 180, 182, 190, 205, 207, 210, and 249.

Embodiment 3. The variant lipolytic enzyme of embodiments 1 and 2, wherein the variant comprises one or more substitutions selected from the group consisting of V014S, R040A/T, G059Y, G061D, T064V, A066D, S07E, Q161H, G175A, R190L, S212D, F226L, A236P, Y239I, L249P, S252I, E254Q, R256K, and L258F, wherein the positions are numbered by reference to the amino acid sequence of SEQ ID NO: 2.

Embodiment 4. The variant lipolytic enzyme of any of the preceding embodiments, wherein the variant comprises an amino acid sequence having at least 75%, 80%, 85%, 90%, 91%, 92%, 93%, 94%, 95%, 96%, 97%, 98%, or 99% identity to the full length amino acid sequence of SEQ ID NO: 2.

Embodiment 5. A variant lipolytic enzyme comprising an amino acid sequence having at least 70% identity to the full length amino acid sequence of SEQ ID NO: 2, wherein the lipase variant comprises one, two, three, four, or more substitutions selected from the group consisting of V014S, R040A/T, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, R256K, and L258F, wherein the positions are numbered by reference to the amino acid sequence of SEQ ID NO: 2.

Embodiment 6. The variant lipolytic enzyme of embodiment 5, wherein the variant has esterase activity.

Embodiment 7. The variant lipolytic enzyme of embodiments 5 or 6, wherein the variant comprises an amino acid sequence having at least 75%, 80%, 85%, 90%, 91%, 92%, 93%, 94%, 95%, 96%, 97%, 98%, or 99% identity to the full length amino acid sequence of SEQ ID NO: 2.

Embodiment 8. The variant lipolytic enzyme of any of the preceding embodiments, wherein the variant comprises the substitutions, or combination of substitutions, selected from the group consisting of R040A, G059Y, G061D, T064V, A066D, S07E, Q161H, F180P, Y182A/L, R190L, S205G, S212D, F226L, A236P, Y239I, L249P, S252I, E254Q, R256K, L258F, G061D-Y182A, G061D-S205G, F180P-Y182A, F180P-S205G, Y182A-S205G, V014S-R040A, V014S-R256K, R040A-E254Q, R040A-S205G, G059Y-A236P, G059Y-E254Q, R040A-F180P, G061D-F226L, G061D-R256K, T064V-F226L, A066D-F226L, A066D-L249P, S070E-L258F, T117L-S205G, T117L-L249P, Y182A-L249P, Q161H-Y239I, G175A-A236P, F180P-S205G, F180P-S252I, Y182A-S212D, Y182A-L258F, R190L-S205G, R190L-L249P, S205G-F207T, S205G-F226L, F207T-R256K, S212D-L249P, S212D-E254Q, F226L-R256K, Y239I-L249P, V014S-R040A-F180P, V014S-S205G-R256K, R040A-F226L-E254Q, G059Y-T117L-A236P, G059Y-A236P-E254Q, G059Y-L249P-L258F, G059Y-G061D-F226L, G061D-R256K-L258F, T064V-F226L-S252I, A066D-F226L-R256K, A066D-Y239I-L249P, S070E-S212D-L258F, T117L-S205G-F226L, T117L-F226L-A236P, T117L-S205G-S252I, Q161H-Y182A-A236P, Q161H-Y239I-L249P, G059Y-G175A-F180P, G175A-A236P-L258F, T064V-T177R-F180P, F180P-S205G-Y239I, R040A-F180P-S252I, T117L-Y182A-S212D, Y182A-F226L-L258F, T117L-R190L-L249P, S205G-F207T-A236P, S205G-F226L-L249P, G059Y-F207T-R256K, T064V-S212D-L249P, R040A-S212D-E254Q, F226L-S252I-R256K, Y239I-L249P-R256K, T177R-F180P-S205G, R040A-F180P-S205G, T064V-F180P-S205G, F180P-S205G-L249P, F180P-R190L-S205G, G061D-Y182A-S205G, F180P-Y182A-S205G, G061D-F180P-Y182A-S205G, and G061D-T117L-I178L-F180P-Y182A-R190L-S205G-F207T-S212D-F226L-Y239I-L249P-S252I-L258F, wherein the positions are numbered by reference to the amino acid sequence of SEQ ID NO: 2.

Embodiment 9. The variant lipolytic enzyme of any of the preceding embodiments, wherein the variant comprises the substitutions, or combination of substitutions, selected from the group consisting of R40T-T177N-F180P-S205G, V025T-R40T-T177N-F180P-S205G, R40T-T064V-T177N-F180P-S205G, R40T-T177N-F180P-Y182L-S205G, R40T-T177N-F180P-R190L-S205G, R40T-T177N-F180P-S205G-L249P, R40T-T064V-T177N-F180P-Y182L-S205G, R40T-T064V-T177N-F180P-R190L-S205G, R40T-T064V-T177N-F180P-S205G, R40T-T177N-F180P-Y182L-R190L-S205G, R40T-T177N-F180P-Y182L-S205G-L249P, R40T-T177N-F180P-R190L-S205G-L249P, R040T-T064V-T177N-F180P-Y182L-R190L-S205G-L249P, V025T-R40T-T064V-T177N-F180P-Y182L-R190L-S205G-L249P, R040T-T064V-T177N-F180P-Y182L-R190L-S205G-L249P, R040T-T064V-T177N-F180P-Y182A-R190L-S205G-L249P, V25T-R040T-T064V-T177N-F180P-Y182L-R190L-S205G-L249P, R040T-T064V-T177N-F180P-Y182L-R190L-S205G-L249P, R040T-T064V-T117L-T177N-I178L-F180P-Y182A-R190L-S205G-F207T-S212D-F226L-Y239I-L249P-S252I-L258F, R040A-T064V-T117L-T177N-I178L-F180P-Y182A-R190L-S205G-F207T-S212D-F226L-Y239I-L249P-S252I-L258F, R040T-T064V-T117L-T177R-I178L-F180P-Y82A-R190L-S205G-F207T-S212D-F226L-Y239I-L249P-S252I-L258F, wherein the positions are numbered by reference to the amino acid sequence of SEQ ID NO: 2.

Embodiment 10. A variant lipolytic enzyme, or an active fragment thereof, comprising:
(a) an amino acid sequence having at least 70% identity to the full-length amino acid sequence of SEQ ID NO: 2,
(b) two or more substitutions at positions selected from
   i) a substitution at position 61 and at least one additional substitution from a position selected from: 14, 40, 59, 64, 66, 70, 161, 175, 180, 182, 190, 205, 212, 226, 236, 239, 249, 252, 254, 256, and 258,
   ii) a substitution at position 180 and at least one additional substitution at a position selected from 14, 40, 59, 61, 64, 66, 70, 161, 175, 182, 190, 205, 212, 226, 236, 239, 249, 252, 254, 256, and 258;
   iii) a substitution at position 182 and at least one additional substitution at a position selected from 14, 40, 59, 61, 64, 66, 70, 161, 175, 180, 190, 205, 212, 226, 236, 239, 249, 252, 254, 256, and 258; or
   iv) a substitution at position 205 and at least one additional substitution at a position selected from 40, 59, 61, 64, 66, 70, 161, 180, 182, 190, 212, 226, 236, 239, 249, 252, 254, 256, and 258;
   wherein the positions are numbered by reference to the amino acid sequence of SEQ ID NO: 2, and wherein the variant lipolytic enzyme has esterase activity.

Embodiment 11. A variant lipolytic enzyme, or an active fragment thereof, comprising:
(a) an amino acid sequence having at least 70% identity to the full-length amino acid sequence of SEQ ID NO: 2,
(b) a combination of mutations selected from the group consisting of
   (i) a combination of mutations G061D-Y182A/L and at least one additional mutation selected from the group consisting of V014S, R040A/T, G059Y, T064V, A066D, S07E, T117L, Q161H, G175A, T177N/R, I178L, F180P, R190L, S205G, F207T, V210I, S212D, F226L, A236P, Y239I, L249P, S252I, E254Q, R256K, and L258F,
   (ii) a combination of mutations G061D-S205G and at least one additional mutation selected from the group consisting of V014S, R040A/T, G059Y, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
   (iii) a combination of mutations F180P-Y182A/L and at least one additional mutation selected from the group consisting of V014S, R040A/T, G059Y, G061D, T064V, A066D, S07E, T117L, Q161H, G175A, T177N/R, I178L, R190L, S205G, F207T, V210I, S212D, F226L, A236P, Y239I, L249P, S252I, E254Q, R256K, and L258F,
   (iv) a combination of mutations F180P-S205G and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F,
   (v) a combination of mutations Y182A/L-S205G and at least one additional mutation selected from the group consisting of V014S, R040A/T, G059Y, G061D, T064V, A066D, S07E, T117L, Q161H, G175A, T177N/R, I178L, F180P, R190L, F207T, V210I, S212D, F226L, A236P, Y239I, L249P, S252I, E254Q, R256K, and L258F,
   (vi) a combination of mutations V014S-R040A and at least one additional mutation selected from the group consisting of G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, R256K, and L258F,
   (vii) a combination of mutations V014S-R256K and at least one additional mutation selected from the group consisting of R040A/T, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
   (viii) a combination of mutations R040A-E254Q and at least one additional mutation selected from the group consisting of V014S, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
   (ix) a combination of mutations R040A-S205G and at least one additional mutation selected from the group consisting of V014S, G059Y, G061D, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
   (x) a combination of mutations G059Y-A236P and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, Y239I, L249P, S252I, E254Q, R256K, and L258F,
   (xi) a combination of mutations G059Y-E254Q and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
   (xii) a combination of mutations R040A-F180P and at least one additional mutation selected from the group consisting of V014S, G059Y, G061D, T064V, A066D, S07E, T117L, Q161H, G175A, T177N/R, I178L, Y182A/L, R190L, S205G, F207T, V210I, S212D, F226L, A236P, Y239I, L249P, S252I, E254Q, R256K, and L258F,
   (xiii) a combination of mutations G061D-F226L and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
   (xiv) a combination of mutations G061D-R256K and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
   (xv) a combination of mutations T064V-F226L and at least one additional mutation selected from the group consisting of V014S, R040A/T, G059Y, G061D, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
   (xvi) a combination of mutations A066D-F226L and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
   (xvii) a combination of mutations A066D-L249P and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, F226L, A236P, Y239I, S252I, E254Q, R256K, and L258F,
   (xviii) a combination of mutations S070E-L258F and at least one additional mutation selected from the group consisting of V014S, R040A/T, G059Y, G061D, T064V, A066D, T117L, Q161H, G175A, T177N/R, I178L, F180P, Y182A/L, R190L, S205G, F207T, V210I, S212D, F226L, A236P, Y239I, L249P, S252I, E254Q, and R256K,
   (xix) a combination of mutations T117L-S205G and at least one additional mutation selected from the group consisting of V014S, R040A/T, G059Y, G061D, T064V, A066D, S07E, Q161H, G175A, T177N/R, I178L, F180P, Y182A/L, R190L, F207T, V210I, S212D, F226L, A236P, Y239I, L249P, S252I, E254Q, R256K, and L258F,
   (xx) a combination of mutations T117L-L249P and at least one additional mutation selected from the group consisting of V014S, R040A/T, G059Y, G061D, T064V, A066D, S07E, Q161H, G175A, T177N/R, I178L, F180P, Y182A/L, R190L, S205G, F207T, V210I, S212D, F226L, A236P, Y239I, S252I, E254Q, R256K, and L258F,
   (xxi) a combination of mutations Y182A/L-L249P and at least one additional mutation selected from the group consisting of V014S, R040A/T, G059Y, G061D, T064V, A066D, S07E, T117L, Q161H, G175A, T177N/R, I178L, F180P, R190L, S205G, F207T, V210I, S212D, F226L, A236P, Y239I, S252I, E254Q, R256K, and L258F,
   (xxii) a combination of mutations Q161H-Y239I and at least one additional mutation selected from the group consisting of V014S, R040A/T, G059Y, G061D, T064V, A066D, S07E, T117L, G175A, T177N/R, I178L, F180P, Y182A/L, R190L, S205G, F207T, V210I, S212D, F226L, A236P, L249P, S252I, E254Q, R256K, and L258F, and
   (xxiii) a combination of mutations G175A-A236P and at least one additional mutation selected from the group consisting of V014S, R040A/T, G059Y, G061D, T064V, A066D, S07E, T117L, Q161H, T177N/R, I178L, F180P, Y182A/L, R190L, S205G, F207T, V210I, S212D, F226L, Y239I, L249P, S252I, E254Q, R256K, and L258F, wherein the positions are numbered by reference to the amino acid sequence of SEQ ID NO: 2, and wherein the variant lipolytic enzyme has esterase activity.

Embodiment 12. A variant lipolytic enzyme comprising:
(a) an amino acid sequence having at least 70% identity to the full length amino acid sequence of SEQ ID NO: 2,
(b) a combination of mutations selected from the group consisting of
   (i) T177N/R-F180P-S205G and at least one additional mutation selected from the group consisting of V014S, R040A/T, G059Y, G061D, T064V, A066D, S07E, T117L, Q161H, G175A, I178L, Y182A/L, R190L, F207T, V210L, S212D, F226L, A236P, Y239I, L249P, S252I, E254Q, R256K, and L258F;
   (ii) R040A/T-F180P-S205G and at least one additional mutation selected from the group consisting of V014S, G059Y, G061D, T064V, A066D, S07E, T117L, Q161H, G175A, T177N/R I178L, Y182A/L, R190L, F207T, V210I, S212D, F226L, A236P, Y239I, L249P, S252I, E254Q, R256K, and L258F;
   (iii) G059Y-F180P-S205G and at least one additional mutation selected from the group consisting of V014S, R040A/T, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
   (iv) G061D-F180P-S205G and at least one additional mutation selected from the group consisting of V014S, R040A/T, G059Y, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
   (v) T064V-F180P-S205G and at least one additional mutation selected from the group consisting of V014S, R040A/T, G059Y, G061D, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
   (vi) A066D-F180P-S205G and at least one additional mutation selected from the group consisting of V014S, R040A/T, G059Y, G061D, T064V, S07E, T117L, Q161H, G175A, T177N/R, I178L, Y182A/L, R190L, F207T, V210I, S212D, F226L, A236P, Y239I, L249P, S252I, E254Q, R256K, and L258F;
   (vii) S070E-F180P-S205G and at least one additional mutation selected from the group consisting of V014S, R040A/T, G059Y, G061D, T064V, A066D, T117L, Q161H, G175A, T177N/R, I178L, Y182A/L, R190L, F207T, V210I, S212D, F226L, A236P, Y239I, L249P, S252I, E254Q, R256K, and L258F;
   (viii) T117L-F180P-S205G and at least one additional mutation selected from the group consisting of V014S, R040A/T, G059Y, G061D, T064V, A066D, S07E, Q161H, G175A, T177N/R, I178L, Y182A/L, R190L, F207T, V210I, S212D, F226L, A236P, Y239I, L249P, S252I, E254Q, R256K, and L258F;
   (ix) Q161H-F180P-S205G and at least one additional mutation selected from the group consisting of V014S, R040A/T, G059Y, G061D, T064V, A066D, S07E, T117L, G175A, T177N/R, I178L, Y182A/L, R190L, F207T, V210I, S212D, F226L, A236P, Y239I, L249P, S252I, E254Q, R256K, and L258F;
   (x) I178L-F180P-S205G and at least one additional mutation selected from the group consisting of V014S, R040A/T, G059Y, G061D, T064V, A066D, S07E, T117L, Q161H, G175A, T177N/R, Y182A/L, R190L, F207T, V210I, S212D, F226L, A236P, Y239I, L249P, S252I, E254Q, R256K, and L258F;
   (xi) F180P-Y182A/L-S205G and at least one additional mutation selected from the group consisting of V014S, R040A/T, G059Y, G061D, T064V, A066D, S07E, T117L, Q161H, G175A, T177N/R, I178L, R190L, F207T, V210L, S212D, F226L, A236P, Y239I, L249P, S252I, E254Q, R256K, and L258F;
   (xii) F180P-R190L-S205G and at least one additional mutation selected from the group consisting of V014S, R040A/T, G059Y, G061D, T064V, A066D, S07E, T117L, Q161H, G175A, T177N/R, I178L, Y182A/L, F207T, V210I, S212D, F226L, A236P, Y239I, L249P, S252I, E254Q, R256K, and L258F;
   (xiii) F180P-S205G-F207T and at least one additional mutation selected from the group consisting of V014S, R040A/T, G059Y, G061D, T064V, A066D, S07E, T117L, Q161H, G175A, T177N/R, I178L, Y182A/L, R190L, V210I, S212D, F226L, A236P, Y239I, L249P, S252I, E254Q, R256K, and L258F;
   (xiv) F180P-S205G-V210I and at least one additional mutation selected from the group consisting of V014S, R040A/T, G059Y, G061D, T064V, A066D, S07E, T117L, Q161H, G175A, T177N/R, I178L, Y182A/L, R190L, F207T, S212D, F226L, A236P, Y239I, L249P, S252I, E254Q, R256K, and L258F;
   (xv) F180P-S205G-S212D and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, F226L, A236P, Y239I, L249P, S252I, E254Q, R256K, and L258F;
   (xvi) F180P-S205G-F226L and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, A236P, Y239I, L249P, S252I, E254Q, R256K, and L258F;
   (xvii) F180P-S205G-A236P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, Y239I, L249P, S252I, E254Q, R256K, and L258F;
   (xviii) F180P-S205G-Y239I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, L249P, S252I, E254Q, R256K, and L258F;
   (xix) F180P-S205G-L249P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, S252I, E254Q, R256K, and L258F;
   (xx) F180P-S205G-S252I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, E254Q, R256K, and L258F;
   (xxi) F180P-S205G-E254Q and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, R256K, and L258F;
   (xxii) F180P-S205G-R256K and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and L258F; and
   (xxiii) F180P-S205G-L258F and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and R256K

wherein the positions are numbered by reference to the amino acid sequence of SEQ ID NO: 2, and wherein the variant lipolytic enzyme has esterase activity.

Embodiment 13. The variant lipolytic enzyme of embodiments 11 or 12, wherein the variant comprises an amino acid sequence having at least 75%, 80%, 85%, 90%, 91%, 92%, 93%, 94%, 95%, 96%, 97%, 98%, or 99% identity to the full length amino acid sequence of SEQ ID NO: 2.

Embodiment 14. The variant lipolytic enzyme of and of embodiments 11-13, wherein the variant comprises a combination of mutations selected from the group consisting of
R040T-T064V-T177N-F180P-Y182L-R190L-S205G-L249P, and
R040T-T064V-T117L-T177N-I178L-F180P-Y182A-R190L-S205G-F207T-S212D-F226L-Y239I-L249P-S252I-L258F,
wherein the positions are numbered by reference to the amino acid sequence of SEQ ID NO: 2.

Embodiment 15. The variant lipolytic enzyme of any of the preceding embodiments, wherein the variant has one or more improved properties when compared to a parent or reference lipolytic enzyme, wherein the improved property is selected from improved stability, improved hydrolytic activity on a polyester, or combinations thereof.

Embodiment 16. The variant lipolytic enzyme of any of embodiment 15, wherein the improved property is:
(i) improved stability, wherein said variant has a residual activity at least 5% when measured in accordance with the stability assay of Example 2 and/or
   (ii) improved hydrolytic activity on a polyester, wherein said variant has a PI≥1.1 compared to the lipase having the amino acid sequence of SEQ ID NO: 2 when measured in accordance with the PET assay of Example 2 or 3.

Embodiment 17. A polynucleotide comprising a nucleic acid sequence encoding a variant lipolytic enzyme of any one of embodiments 1-16.

Embodiment 18. The polynucleotide of embodiment 17, wherein the nucleic acid sequence is operably linked to a promoter.

Embodiment 19. An expression vector or cassette comprising the polynucleotide of embodiment 17 or 18.

Embodiment 20. A recombinant host cell comprising the expression vector or cassette of embodiment 19.

Embodiment 21. An enzyme composition comprising a variant lipolytic enzyme of any one of embodiments 1-16.

Embodiment 22. A cleaning composition or detergent composition comprising a variant lipolytic enzyme of any one of embodiments 1-16 and at least one adjunct selected from the group consisting of surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

Embodiment 23. The cleaning or detergent composition of embodiment 22, wherein the composition further comprises at least one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, lysozymes, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

Embodiment 24. A fabric treatment composition comprising a variant lipolytic enzyme of any one of embodiments 1-16 and at least one adjunct selected from the group consisting of surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

Embodiment 25. The fabric treatment composition of embodiment 24, wherein the composition further comprises at least one additional enzyme selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, lysozymes, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

Embodiment 26. A method for treating a fabric or textile comprising, (i) contacting a fabric or textile with a variant lipolytic enzyme of any one of embodiments 1-16 or a composition comprising said variant lipolytic enzyme, and (ii) optionally, rinsing the fabric or textile.

Embodiment 27. The method of embodiment 26, wherein the fabric or textile comprises at least one polyester.

Embodiment 28. The method of any of embodiments 26 and 27, wherein the contacting step comprises a variant lipolytic enzyme in an amount selected from the group consisting of
i) 0.002 to 10,000 mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or
ii) in the amount of at least 0.01 ppm active enzyme.

Embodiment 29. A method for degrading a polyester or a polyester containing material comprising
i) contacting the polyester containing material with a variant lipolytic enzyme according to any one of embodiments 1-16 or a composition of embodiments 22-25, and, optionally,
ii) rinsing said polyester containing material.

Embodiment 30. A method for the enzymatic depolymerization of a polyester or a polyester containing material comprising,
i) contacting the polyester or polyester containing material with a variant lipolytic enzyme according to any one of embodiments 1-16 or a composition of embodiments 22-25, and, optionally,
ii) recovering monomers and/or oligomers of the polyester.

Embodiment 31. The method of embodiment 29 or 30, wherein the polyester is selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

Embodiment 32. The lipolytic enzyme of any of embodiments 1-16, wherein the variant has lypolytic activity on a polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

### EXAMPLES

### Example 1

### Recombinant expression of P. mendocina lipase variants

A synthetic, codon-optimized gene (SEQ ID NO:1) encoding the wild-type Pseudomonas mendocina lipase (SEQ ID NO:2) was made and served as template for the construction of plasmids expressing the WT and variant polypeptides thereof. In this study, a Pseudomonas mendocina lipase variant with substitutions R040T, T177N, F180P, and S205G was also used as a starting point in the engineering of further substituted variants and is referred to as PEV001. Lipase genes were produced by either GeneArt AG (Regensburg, Germany) or Twist Bioscience (San Francisco, U.S.A.) and cloned into the pSB expression vector (Babe, L. M., et al. (1998) BiotechnolApplBiochem. 27:117-24) using standard molecular biology techniques resulting in expression plasmids suitable for expression in Bacillus subtilis. Elements of the constructs included: DNA fragments comprising the aprE promoter sequence (SEQ ID NO:3), the nucleotide sequence encoding either the aprE signal peptide sequence (SEQ ID NO:4) or a hybrid aprE-P. mendocina lipase signal peptide sequence (SEQ ID NO:5), the sequence corresponding to the gene encoding a mature lipase, the BPN′ terminator (SEQ ID: NO:6), and additional elements from pUB110 (McKenzie et al. (1986) Plasmid 15: 93-103) including a replicase gene (reppUB), a neomycin/kanamycin resistance gene (neo), and a bleomycin resistance marker (bleo).

A suitable B. subtilis host strain was transformed with the pSB expression plasmid using a method known in the art (WO 02/14490). The transformation mixtures were plated onto LA plates containing 10 ppm neomycin sulfate and incubated overnight at 37° C. Single colonies were picked and grown in Luria broth at 37° C. under antibiotic selection.

### Example 2

### Generation and Characterization of P. mendocina lipase variants

For protein expression experiments, transformed cells were grown in 96-well MTPs in cultivation medium (enriched semi-defined media based on MOPs buffer, with urea as major nitrogen source, glucose as the main carbon source, and supplemented with 1% soytone for robust cell growth) containing 10 ppm neomycin for 3 days at 32° C., 270 rpm, with 80% humidity in a shaking incubator.

Cultures grown in MTP were harvested by centrifugation at 3600 rpm for 15 min and filtered through Multiscreen® filter plates (EMD Millipore, MA, USA) using a Millipore vacuum system. The filtered culture supernatants were used for the assays described below. Enzyme concentration was determined using pNB substrate hydrolysis assay. 1 mM pNB substrate (4-nitrophenyl butyrate, Catalog #N9876, Sigma) was prepared by adding 0.2 mL of pNB stock solution (100 mM in DMSO) to 20 mL of buffer (100 mM Tris-HCl, 0.1% Triton X-100, pH8). 10 ul of diluted enzyme was added into 190 ul of 1 mM pNB in assay buffer in 96 well plate (Costar, #9017, ThermoFisher) to start the reaction. The plate was mixed well on the plate shaker and OD 405 nm was monitored every 12 seconds for 3 minutes in Microplate reader (Molecular devices, SpectraMax plus 384). The Vmax in mOD/min value of a sample not containing enzyme (blank) was subtracted from Vmax values of the enzyme-containing samples. The resulting Vmax in mOD/min was reported as enzyme concentration.

Enzyme Activity Assay on PET substrate

The enzymatic activity of variants was tested by measuring the hydrolysis of PET Pellet (Polyethylene terephthalate) substrate in solution. PET pellets were purchased from Scientific Polymer Products (Cat #138). One PET pellet (20-30 mg) was added to each well of a microtiter plate (Nunc, 267245) and detergent solutions were added as follows: 150 ul of Formula A HDL (3.5 g/L) (composition in Table 1) with 11 mM Tris-HCl buffer with 7 gpg water hardness Ca:Mg=3:1, pH 8, or 150 ul of Tide® Original detergent (Tide, manufactured by Procter and Gamble, purchased in 2020) (0.875 g/L) with 11 mM Tris-HCl and 7 gpg water Hardness Ca:Mg=3:1. A set of plates without PET in the well were also set up to serve as controls for enzyme background. 20 ul of each enzyme sample was added per well of the assay plate to initiate the reaction. The reaction was carried out at 28° C. for 24 hours with shaking (180 rpm) in incubation shaker (Infors HT, Multitron). After incubation, 100 ul of the reaction supernatant was transferred into a new UV-transparent plate (Corning 3635) and measured at 240 nm on Microplate reader (Molecular devices, SpectraMax plus 384). The resulting absorbance after subtracting absorbance from enzyme background plate was taken as a measure of PET hydrolysis activity. The absorbance values were plotted against enzyme concentration. Each variant was assayed in triplicates. PET activity is reported as Performance Index (PI) values, which were calculated by dividing the PET activity of each variant by that of the parent, tested at the same protein concentration. Theoretical values for the PET activity of the parent enzyme at the relevant protein concentrations were calculated using the parameters extracted from a Langmuir fit of measured values from a standard curve of the parent enzyme activity

TABLE 1
Composition of Custom-made Formula A HDL Detergent

| | WT % of active | WT % of active |
|---|---|---|
| | substance in the | substance in the |
| Chemical name | raw material | formulation |
| Water demin. | 100 | Ad 100 |
| Alkyl benzene sulfonic acid | 96 | 3-7 |
| (LAS) | | |
| Anionic surfactant (FAEOS) | 70 | 2-6 |
| Coconut fatty acid | 30 | 0.3-1 |
| Non-ionic surfactant (FAEO) | 100 | 3-7 |
| Citric acid | 100 | 0.1-2 |
| NaOH | 50 | 0.3-1 |
| Glycerol | 99.5 | 0.3-1 |
| Preserving agent | 100 | 0.05-0.1 |
| Boric acid | 100 | 0.3-1 |
| Optical Brightener | 90 | 0.01-0.08 |
| Thickener | 25 | 1-3 |
| Enzymes (except lipase) | 100 | 0.5-2 |
| Dequest | 40 | 0.1-2 |
| Dye, Perfume, Antifoam | t.q. | minors |
| pH = 8.2 to 8.6 | | |
| Dosage = 50 mL | | |

### Thermal Stability of Enzyme Variants

The stability of enzyme variants was tested under stress condition in a 25% (v/v) aqueous solution of Formula A HDL detergent by measuring the residual activity of samples after incubation at elevated temperature 48° C. for one hour. A 50% (v/v) aqueous solution of the detergent was prepared and enzyme samples from filtered culture supernatants were mixed with the appropriate volume of this detergent solution to achieve 25% (v/v) final detergent concentration. For detergent stability assays using Tide® Original detergent, enzyme samples from filtered culture supernatants were mixed with the appropriate volume of detergent solution to achieve 16% (v/v) final detergent concentration. To measure the initial (unstressed) activity, aliquots of this mixture were immediately diluted in 100 mM Tris-HCl, 0.1% Triton X-100, pH8 and assayed for activity on pNB substrate. pNB substrate (4-nitrophenyl butyrate, Sigma) solution (1 mM) was prepared by adding 0.2 mL of pNB stock solution (100 mM in DMSO) to 20 mL of buffer (100 mM Tris-HCl, 0.1% Triton X-100, pH8). 10 ul of diluted enzyme solution was mixed into 190 ul of 1 mM pNB in assay buffer in 96 well plate (Costar, #9017, ThermoFisher) to start the reaction. The plate was mixed thoroughly, and absorbance was monitored at OD 405 nm every 12 seconds for 3 minutes in a Microplate reader (Molecular devices, SpectraMax plus 384). The Vmax in mOD/min value of a sample not containing enzyme (blank) was subtracted from Vmax values of the enzyme-containing samples. The resulting Vmax in mOD/min was recorded as enzyme activity on pNB substrate. Once stressed and unstressed activity values were measured by hydrolysis of pNB substrate as described above, the percent (%) residual activities were calculated by taking a ratio of the stressed to unstressed activity and multiplying by 100. Table 2 shows the polyesterase activity on PET (performance index) and stability (% residual activity) of P. mendocina lipase variants.

TABLE 2
Stability and activity of P. mendocina lipase variants in liquid detergent.
Stability is reported as % residual activity remaining after incubation at
48° C. for 1 hour. Relative activity on PET substrate is reported as Performance
Index values (PI) calculated relative to P. mendocina lipase WT.

| | Amino acid | | | | |
|---|---|---|---|---|---|
| | substitution | | | Polyesterase Activity | |
| | with respect to | % Residual activity | | (Performance Index) | |
| | P. mendocina | Formula | Tide | Formula | Tide |
| sample ID | WT (parent) | A HDL | HDL | A HDL | HDL |
| WT | none | 0 | 0 | 1.0 | 1.0 |
| Variant 13 | R040A | 5% | 41% | 1.4 | 1.1 |
| Variant 14 | G059Y | 6% | >100% | 0.7 | 0.7 |
| Variant 15 | G061D | 6% | >100% | 1.6 | 1.5 |
| Variant 16 | T064V | 7% | 72% | 1.7 | 1.4 |
| Variant 17 | A066D | 9% | 55% | 1.7 | 1.6 |
| Variant 18 | S070E | 6% | 74% | 1.4 | 1.5 |
| Variant 20 | Q161H | 6% | 6% | 1.4 | 1.3 |
| Variant 24 | F180P | 18% | 21% | 3.3 | 2.8 |
| Variant 25 | Y182A | 12% | >100% | 1.2 | 0.8 |
| Variant 26 | R190L | 17% | 21% | 1.6 | 1.0 |
| Variant 27 | S205G | 16% | 47% | 0.7 | 0.6 |
| Variant 30 | S212D | 15% | >100% | 1.8 | 1.5 |
| Variant 31 | F226L | 4% | 19% | 1.7 | 1.7 |
| Variant 32 | A236P | 3% | 12% | 1.7 | 1.7 |
| Variant 33 | Y239I | 4% | 85% | 1.6 | 1.4 |
| Variant 34 | L249P | 12% | 58% | 1.3 | 1.5 |
| Variant 35 | S252I | 6% | >100% | 1.3 | 1.0 |
| Variant 36 | E254Q | 4% | >100% | 1.3 | 1.1 |
| Variant 37 | R256K | 12% | 84% | 1.5 | 1.2 |
| Variant 38 | L258F | 5% | 9% | 1.7 | 1.7 |
| Variant 2 | G061D-Y182A | 11% | 7% | 2.2 | 2.4 |
| Variant 3 | G061D-S205G | 17% | 0% | 2.2 | 1.8 |
| Variant 4 | F180P-Y182A | 19% | 12% | 4.3 | 4.0 |
| Variant 5 | F180P-S205G | 23% | 28% | 4.0 | 3.7 |
| Variant 6 | Y182A-S205G | 21% | 92% | 1.4 | 1.1 |
| Variant 39 | V014S-R040A | 5% | 83% | 1.3 | 1.3 |
| Variant 40 | V014S-R256K | 11% | >100% | 1.2 | 1.1 |
| Variant 41 | R040A-E254Q | 13% | >100% | 0.7 | 0.3 |
| Variant 42 | R040A-S205G | 14% | 47% | 1.6 | 1.5 |
| Variant 43 | G059Y-A236P | 2% | 52% | 0.7 | 0.7 |
| Variant 44 | G059Y-E254Q | 10% | 54% | 0.6 | 0.6 |
| Variant 45 | R040A-F180P | 19% | 4% | 2.7 | 2.9 |
| Variant 46 | G061D-F226L | 8% | 7% | 1.9 | 2.0 |
| Variant 47 | G061D-R256K | 7% | 25% | 1.7 | 1.9 |
| Variant 50 | T064V-F226L | 12% | 95% | 1.2 | 1.0 |
| Variant 51 | A066D-F226L | 17% | 58% | 1.9 | 1.8 |
| Variant 51 | A066D-F226L | 14% | 65% | 1.9 | 1.5 |
| Variant 51 | A066D-F226L | 4% | >100% | 4.0 | 3.2 |
| Variant 52 | A066D-L249P | 33% | 80% | 1.5 | 1.4 |
| Variant 54 | S070E-L258F | 18% | 37% | 2.1 | 2.1 |
| Variant 55 | T117L-S205G | 5% | >100% | 0.7 | 0.7 |
| Variant 57 | T117L-L249P | 27% | >100% | 1.1 | 0.6 |
| Variant 58 | Y182A-L249P | 21% | 55% | 1.7 | 1.8 |
| Variant 60 | Q161H-Y239I | 6% | 35% | 1.5 | 1.8 |
| Variant 62 | G175A-A236P | 3% | 22% | 1.7 | 1.7 |
| Variant 67 | F180P-S205G | 24% | 24% | 4.4 | 3.7 |
| Variant 68 | F180P-S252I | 9% | 34% | 4.1 | 3.3 |
| Variant 69 | Y182A-S212D | 16% | 12% | 2.2 | 2.2 |
| Variant 70 | Y182A-L258F | 13% | 11% | 2.0 | 2.0 |
| Variant 71 | R190L-S205G | 25% | 5% | 1.3 | 1.0 |
| Variant 72 | R190L-L249P | 39% | 5% | 1.3 | 1.3 |
| Variant 73 | S205G-F207T | 5% | 2% | 2.1 | 1.6 |
| Variant 74 | S205G-F226L | 14% | 67% | 1.9 | 1.8 |
| Variant 75 | F207T-R256K | 8% | 2% | 2.1 | 1.7 |
| Variant 77 | S212D-L249P | 39% | 44% | 2.2 | 1.9 |
| Variant 78 | S212D-E254Q | 19% | 10% | 1.8 | 1.8 |
| Variant 79 | F226L-R256K | 21% | 44% | 1.4 | 1.2 |
| Variant 80 | Y239I-L249P | 41% | 21% | 1.4 | 1.1 |
| Variant 81 | V014S-R040A-F180P | 14% | 3% | 3.1 | 3.2 |
| Variant 82 | V014S-S205G-R256K | 18% | 31% | 1.0 | 0.9 |
| Variant 83 | R040A-F226L-E254Q | 18% | 17% | 1.3 | 1.2 |
| Variant 85 | G059Y-T117L-A236P | 3% | 23% | 0.7 | 0.8 |
| Variant 86 | G059Y-A236P-E254Q | 17% | 66% | 0.7 | 0.7 |
| Variant 87 | G059Y-L249P-L258F | 45% | >100% | 1.5 | 1.5 |
| Variant 88 | G059Y-G061D-F226L | 2% | 2% | 1.6 | 1.4 |
| Variant 89 | G061D-R256K-L258F | 26% | 7% | 2.5 | 2.4 |
| Variant 92 | T064V-F226L-S252I | 31% | 94% | 1.6 | 1.5 |
| Variant 93 | A066D-F226L-R256K | 31% | 41% | 2.3 | 2.6 |
| Variant 94 | A066D-Y239I-L249P | 36% | 57% | 1.2 | 1.9 |
| Variant 96 | S070E-S212D-L258F | 41% | 17% | 2.1 | 1.9 |
| Variant 97 | T117L-S205G-F226L | 19% | 74% | 1.3 | 1.2 |
| Variant 98 | T117L-F226L-A236P | 6% | 24% | 1.4 | 1.2 |
| Variant 100 | T117L-S205G-S252I | 12% | 28% | 1.0 | 1.0 |
| Variant 101 | Q161H-Y182A-A236P | 10% | 38% | 2.0 | 2.0 |
| Variant 102 | Q161H-Y239I-L249P | 16% | 35% | 2.0 | 2.0 |
| Variant 103 | G059Y-G175A-F180P | 9% | 5% | 3.7 | 3.4 |
| Variant 104 | G175A-A236P-L258F | 18% | 46% | 1.4 | 1.2 |
| Variant 105 | T064V-T177R-F180P | 43% | 73% | 1.1 | 1.1 |
| Variant 109 | F180P-S205G-Y239I | 30% | >100% | 3.8 | 3.5 |
| Variant 110 | R040A-F180P-S252I | 14% | 13% | 3.5 | 3.9 |
| Variant 111 | T117L-Y182A-S212D | 29% | 22% | 2.0 | 1.9 |
| Variant 112 | Y182A-F226L-L258F | 35% | >100% | 2.0 | 1.7 |
| Variant 114 | T117L-R190L-L249P | 29% | 22% | 1.4 | 1.4 |
| Variant 115 | S205G-F207T-A236P | 6% | 4% | 3.7 | 2.7 |
| Variant 116 | S205G-F226L-L249P | 78% | >100% | 1.4 | 1.3 |
| Variant 117 | G059Y-F207T-R256K | 4% | 5% | 1.6 | 1.3 |
| Variant 119 | T064V-S212D-L249P | 38% | 28% | 1.8 | 2.1 |
| Variant 120 | R040A-S212D-E254Q | 27% | 46% | 1.7 | 1.8 |
| Variant 121 | F226L-S252I-R256K | 42% | >100% | 1.6 | 1.5 |
| Variant 122 | Y239I-L249P-R256K | 53% | 72% | 1.8 | 1.9 |
| Variant 123 | T177R-F180P-S205G | 72% | >100% | 0.7 | 0.8 |
| Variant 124 | R040A-F180P-S205G | 30% | 97% | 4.5 | 4.4 |
| Variant 125 | T064V-F180P-S205G | 32% | >100% | 4.1 | 3.5 |
| Variant 126 | F180P-S205G-L249P | 44% | 94% | 4.0 | 3.8 |
| Variant 127 | F180P-R190L-S205G | 32% | >100% | 3.9 | 3.4 |
| Variant 9 | G061D-Y182A-S205G | 13% | 54% | 2.8 | 2.5 |
| Variant 10 | F180P-Y182A-S205G | 26% | 60% | 4.7 | 4.6 |
| Variant 11 | G061D-F180P-Y182A-S205G | 35% | 31% | 5.4 | 5.8 |
| Variant 128 | G061D-T117L-I178L-F180P- | 99% | >100% | 11.8 | 7.7 |
| | Y182A-R190L-S205G-F207T- | | | | |
| | S212D-F226L-Y239I-L249P- | | | | |
| | S252I-L258F | | | | |

### Example 3

### Generation and Characterization of Additional P. mendocina Lipase Variants

Additional P. mendocina lipase variants with further amino acid substitutions (shown with respect to WT on Table 4) were constructed and evaluated for polyesterase hydrolysis activity and detergent stability as described below.

### Sample Generation and Enzyme Concentration Determination

To generate the enzyme samples for screening, the transformed B. subtilis cells were grown in 96 well microtiter plates (MTPs, manufacture) at 37° C. for 68 hours in cultivation medium (enriched semi-defined media based on MOPS buffer, with urea as the major nitrogen source, glucose as the main carbon source, and supplemented with 1% soytone for robust cell growth) in each well. Cultures were harvested by centrifugation at 3600 rpm for 15 min and filtered through Multiscreen® filter plates (EMD Millipore, Billerica, MA, USA) using a Millipore vacuum system. The filtered culture supernatants were used for the assays described below. Typically, the culture broth was diluted in 100 mM Tris pH8 in 96 well plate (NUNC, 267245). Enzyme concentration was determined by separation of protein components using a Zorbax 300 SB-C3 column (Agilent) and running a linear gradient of 0.1% Trifluoroacetic acid in water (Buffer A) and 0.1% Trifluoroacetic acid in Acetonitrile (Buffer B) with detection at 220 nm column on UHPLC. The enzyme concentration of the samples was calculated using a standard curve of the purified parent enzyme.

### Enzyme Activity in Detergent, Using PET Substrate

The enzyme activity of P. mendocina WT lipase and variants thereof was tested in the following liquid detergents (HDL): Persil® Small & Mighty Non-Bio Liquid Detergent (PNB, manufactured by Unilever) purchased in 2020, custom-made Test HDL 1 detergent (composition shown on Table 1), Tide® Original detergent, and Formula A HDL detergent (composition shown on Table 3). Before use, Tide® detergent was pre-treated by heating the neat liquid at 95° C. for 16 hours in a water bath to inactivate the enzymes present in the formulation prior to using in the enzyme assays.

TABLE 3
Composition of Custom-made Test HDL 1 Detergent Formula

| | | | Wt. |
|---|---|---|---|
| Component | Ingredient | Trade name | % |
| Solvent | Water (total) | — | 64.89 |
| Surfactant | C12-C15 Pareth-7 | Empilan KCL 7 | 3 |
| | (Sodium) | NANSA SSA F | 7.5 |
| | Dodecylbenzenesulfonate | | |
| | K-Cocoate | NANSA PC 38F | 3 |
| | Sodium Laureth Sulfate | Empicol ESB3/M6 | 9 |
| Builder | Sodium Citrate | Sodium Citrate Tribasic | 3 |
| | | Dihydrate | |
| Liquid | Sorbitol | D-Sorbitol | 0.8 |
| properties/ | Propylene glycol | 1,2-Propanediol | 2.5 |
| stability | Glycerin | Glycerol | 0.8 |
| | Triethanolamine | Triethanolamine | 0.5 |
| | Methylisothiazolinone | 2-Methyl-4-isothiazolin- | 0.01 |
| | | 3-one | |
| | Ethanol | — | 1 |
| Neutralizer | NaOH (4M) | — | 5.27 |

The enzyme activity of multiply substituted variants was determined by measuring the hydrolysis of PET (Polyethylene terephthalate) substrate. The PET powder was purchased from Scientific Polymer Products (Cat #138). and was suspended to 6-12% (w/v) for assays. HDL detergents were prediluted using 10 mM Tris-HCl buffer with 6 gpg water hardness Ca:Mg=3:1, pH 8 to achieve the following concentrations: HDL detergent formula H (3 g/L); PNB (2.7 g/L); Tide (0.75 g/L); and Test HDL 1 (3 g/L) with. While the PET solutions were kept mixing with a magnetic stirrer, 200 μL of the substrate solution was transferred to a microtiter plate (Nunc, 267245) using wide-bore pipet tips. Diluted enzyme samples were prepared in 100 mM Tris-HCl buffer, pH8, and 10 μL of diluted enzyme sample was added to the assay plates to initiate the enzyme reaction. The reactions were carried out at 40° C. for 20 hours with shaking at 200 rpm in incubation shaker (Infors HT, Multitron). After incubation, the reaction supernatants were collected by filtration using filter plates (Corning 3505). 100 μL of each supernatant was transferred into a new UV-transparent plate (Corning 3635) and to measure absorbance at 240 nm on a microplate reader (Molecular devices, SpectraMax plus 384). The resulting absorbance values were used as a measure polyesterase (PET) hydrolysis activity. In some instances, performance index value (PI) were calculated by dividing the PET activity of variants by that of the parent tested at the same protein concentration. Theoretical values for the PET activity of the parent enzyme at the relevant protein concentrations were calculated using the parameters extracted from a Langmuir fit of measured values for a standard curve of the parent enzyme.

### Thermal Stability in Detergent, Using pNB Substrate

The thermal stability of the enzyme variants was tested under stress conditions using pNB (4-nitrophenyl butyrate) as substrate. The assay measured residual activity of samples after incubation at elevated temperature (50° C. to 56° C.) for 16-20 hours using the Formula A HDL detergent. A 66.7% (v/v) aqueous solution of the Formula A detergent was prepared and enzyme samples from filtered culture supernatants were mixed with the appropriate volume of this detergent solution to achieve 50% (v/v) final detergent concentration. To measure the initial (unstressed) activity, aliquots of this mixture were immediately diluted in 100 mM Tris-HCl, 0.1% Triton X-100, pH8 and assayed for activity on pNB substrate. A 1 mM solution of pNB substrate (4-nitropheyl butyrate, Sigma) was prepared by adding 0.2 mL of pNB stock solution (100 mM in DMSO) to 20 mL of buffer (100 mM Tris-HCl, 0.1% Triton X-100, pH8). 10 ul of diluted enzyme sample was added into 190 ul of 1 mM pNB in assay buffer in 96 well plate (Costar, #9017) to start the reaction. The plate was mixed thoroughly and absorbance at 405 nm was monitored every 12 seconds for 3 minutes in a microplate reader (Molecular devices, SpectraMax plus 384). The Vmax value (reported in mOD/min) of a sample not containing enzyme (blank) was subtracted from Vmax values of the lipase-containing samples. The resulting Vmax in mOD/min was reported as pNB activity. Stressed and unstressed activity values were measured and the percent (%) of residual activity was calculated by taking the ratio of the stressed to unstressed activity for each enzyme and multiplying by 100. The stability results are reported as % remaining (residual) activity.

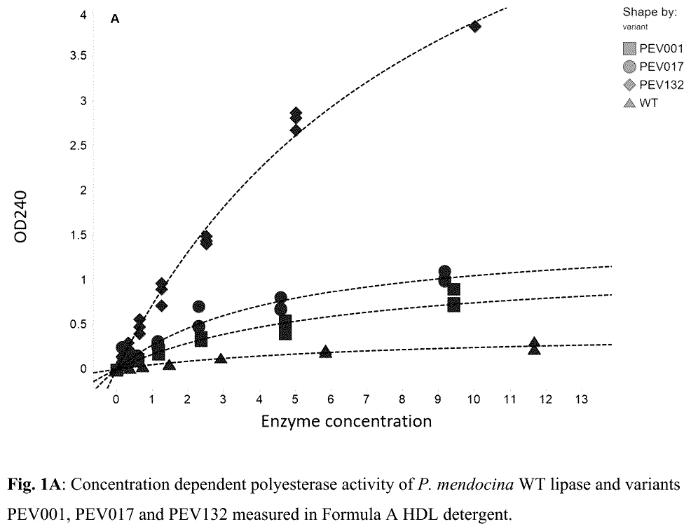

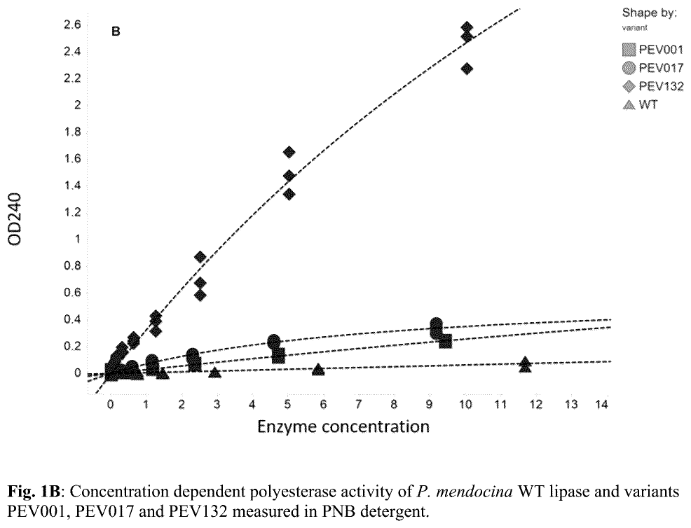

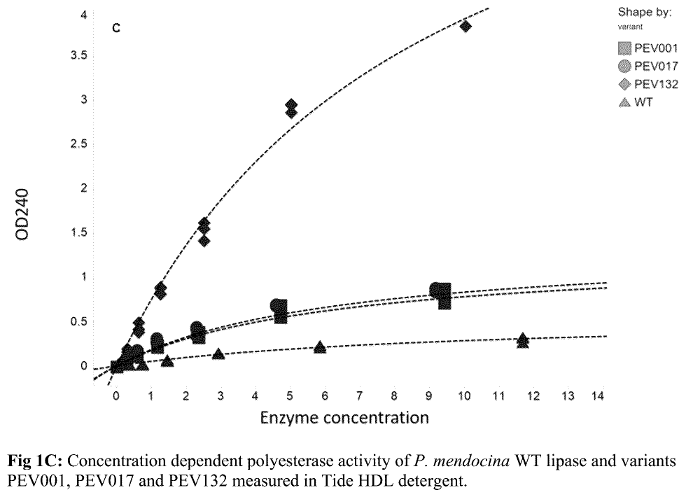

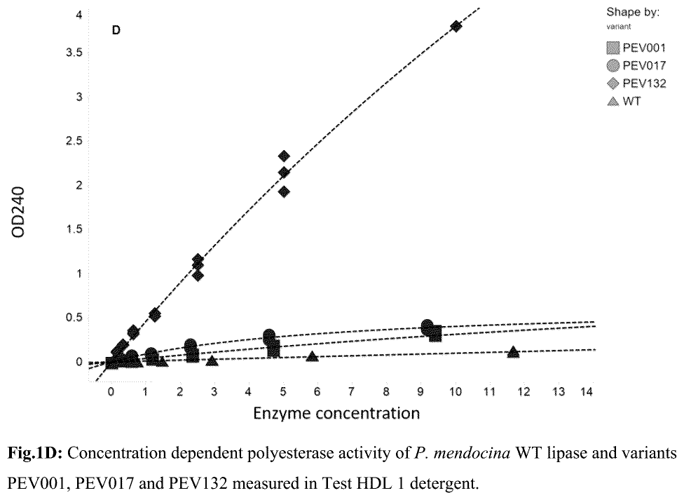

The enzyme activity of P. mendocina WT and variants PEV001, PEV017 and PEV132 (amino acid substitutions with respect to WT shown in Table 4) was measured in the four detergents (Formula A HDL detergent, PNB, Tide®, and Test HDL 1 detergent) as described above. The activity on PET of P. mendocina WT lipase and variants was plotted as absorbance at 240 nm versus enzyme concentration and is shown in FIGS. 1A (Formula A HDL detergent), 1B (PNB); 1C (Tide), 1D (Test HDL 1). The data shows that variants PEV001 and PEV017 have improved PET activity compared to the WT in all detergents tested, and variant PEV132 shows further improvement in PET activity compared to variant PEV017 in all four detergents.

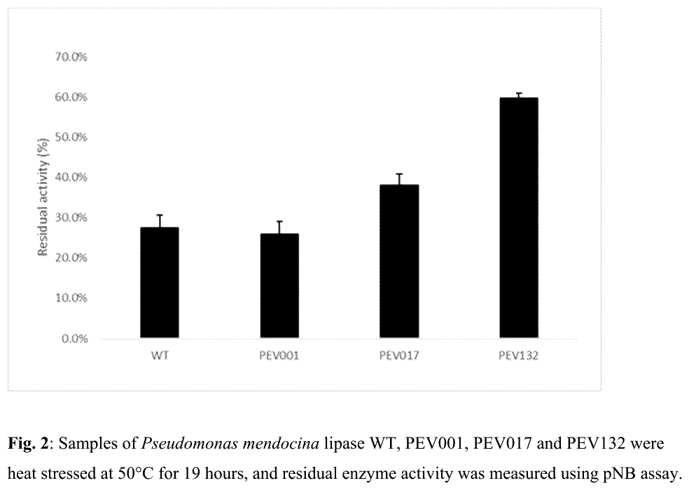

The stability of Pseudomonas mendocina lipase WT and variants PEV001, PEV017 and PEV132 (amino acid substitutions with respect to WT shown in Table 4) was tested under stress conditions in Formula A HDL detergent. Samples were heat stressed at 50° C. for 19 hours and residual activity was measured using the pNB assay as described above. Data shown in FIG. 2 shows that variant PEV017 has improved stability compared to WT lipase and variant PEV132 is further improved in stability compared to PEV017.

FIG. 2: Samples of Pseudomonas mendocina lipase WT, PEV001, PEV017 and PEV132 were heat stressed at 50° C. for 19 hours, and residual enzyme activity was measured using pNB assay.

Table 4 shows the PET hydrolysis (reported as performance index values), and the stability (reported as % remaining activity) of multiply substituted P. mendocina lipase variants. The stability of the variants marked with an * symbol was measured after incubation at 40° C. for 22 hours relative to variant PEVOOL. The stability of variants marked with a ** symbol was measured after incubation at 48° C. for 18 hours relative to variant PEV017. The stability of variants marked with a *** symbol was measured after incubation at 50° C. for 20 hours relative to variant PEV017 (in a separate experiment). Results on Table 4 show the benefit of additional substitutions on the increase in PET activity and or stability in detergent as compared to lesser substituted lipase variants.

TABLE 4
Activity and stability of P. mendocina lipase variants in Formula A HDL detergent.
PET Performance Index values (PI) was calculated relative to PEV001 (*) or PEV017 (**
or ***). Stability is reported as % residual activity remaining after incubation at either
40° C. for 22 hours (*) or 48° C. for 18 hours (**) or 50° C. for 20 hours (***).

| | | PET | Residual |
|---|---|---|---|
| | | activity, | Activity |
| Sample ID | Substitutions with respect to WT | PI | (%) |
| PEV001* | R40T-T177N-F180P-S205G | 1.0 | 46 |
| PEV002* | V025T-R40T-T177N-F180P-S205G | 1.0 | 48 |
| PEV003* | R40T-T064V-T177N-F180P-S205G | 1.0 | 62 |
| PEV004* | R40T-T177N-F180P-Y182L-S205G | 1.0 | 51 |
| PEV005* | R40T-T177N-F180P-R190L-S205G | 1.3 | 54 |
| PEV006* | R40T-T177N-F180P-S205G-L249P | 1.2 | 66 |
| PEV007* | R40T-T064V-T177N-F180P-Y182L-S205G | 1.1 | 66 |
| PEV008* | R40T-T064V-T177N-F180P-R190L-S205G | 1.4 | 62 |
| PEV009* | R40T-T064V-T177N-F180P-S205G | 1.3 | 75 |
| PEV010* | R40T-T177N-F180P-Y182L-R190L-S205G | 1.4 | 49 |
| PEV011* | R40T-T177N-F180P-Y182L-S205G-L249P | 1.3 | 61 |
| PEV012* | R40T-T177N-F180P-R190L-S205G-L249P | 1.4 | 66 |
| PEV017* | R040T-T064V-T177N-F180P-Y182L-R190L-S205G- | 1.6 | 80 |
| | L249P | | |
| PEV018* | V025T-R40T-T064V-T177N-F180P-Y182L-R190L- | 1.4 | 94 |
| | S205G-L249P | | |
| PEV017** | R040T-T064V-T177N-F180P-Y182L-R190L-S205G- | 1.0 | 7.0 |
| | L249P | | |
| PEV017v1** | R040T-T064V-T177N-F180P-Y182A-R190L-S205G- | 1.3 | 12 |
| | L249P | | |
| PEV017v2** | V25T-R040T-T064V-T177N-F180P-Y182L-R190L- | 0.9 | 49 |
| | S205G-L249P | | |
| PEV017*** | R040T-T064V-T177N-F180P-Y182L-R190L-S205G- | 1.0 | 5.0 |
| | L249P | | |
| PEV132*** | R040T-T064V-T117L-T177N-I178L-F180P-Y182A- | 2.7 | 32 |
| | R190L-S205G-F207T-S212D-F226L-Y239I-L249P- | | |
| | S252I-L258F | | |
| PEV132v1*** | R040A-T064V-T117L-T177N-I178L-F180P-Y182A- | 2.9 | 44 |
| | R190L-S205G-F207T-S212D-F226L-Y239I-L249P- | | |
| | S252I-L258F | | |
| PEV132v2*** | R040T-T064V-T117L-T177R-I178L-F180P-Y182A- | 2.8 | 59 |
| | R190L-S205G-F207T-S212D-F226L-Y239I-L249P- | | |
| | S252I-L258F | | |

### Example 4

### Enzyme Performance on Polyester Swatches

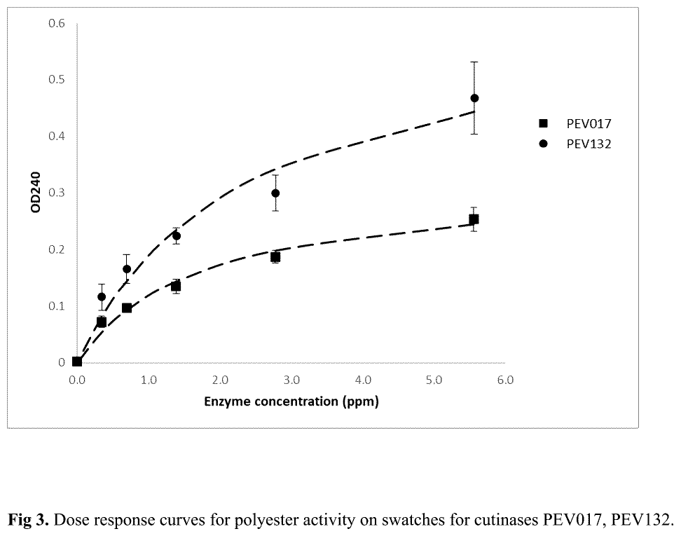

Polyester unsoiled swatches (Testfabric Inc) were punched to fit into wells of 96 well plates (Nunc, polypropylene plate). Formula A HDL detergents were prediluted using 10 mM Tris-HCl buffer with 6 gpg water hardness Ca:Mg=3:1, pH 8 to achieve a of 3 g/L. The plates containing 1 microswatch per well were first filled with 200 ul of Formula A HDL detergent prior to enzyme addition; 10 ul of enzyme of different concentrations in 10 mM Tris-HCl were added to a detergent-filled microswatch plate to reach a final volume of 210 uL with a final enzyme concentration between 0 to 6 ppm. The plates were sealed and were incubated at 40 C for 18 hours with 180 rpm shaking in incubator (Infors HT, Multitron). The enzyme activity was monitored by the release of terephthalate and its esters resulting from the hydrolysis of polyester on the cloth. The soluble terephthalate and its esters have characteristic strong absorbance peaks around 240 nm. Following incubation, 100 uL of supernatant was transferred to a fresh UV-transparent plate (Corning 3635) and absorbance was read at 240 nm using the SpectraMax plate reader. Absorbance results were obtained by subtracting the value for a blank control (no enzyme) from each sample value. Each enzyme dose was tested in triplicates. The dose response curve was fitted with Langmuir fit. FIG. 3 shows the polyester degradation activity in swatches for PEV017, PEV132.

### Example 5

### Structure-Function Comparison of Various P. mendocina Lipase Residues

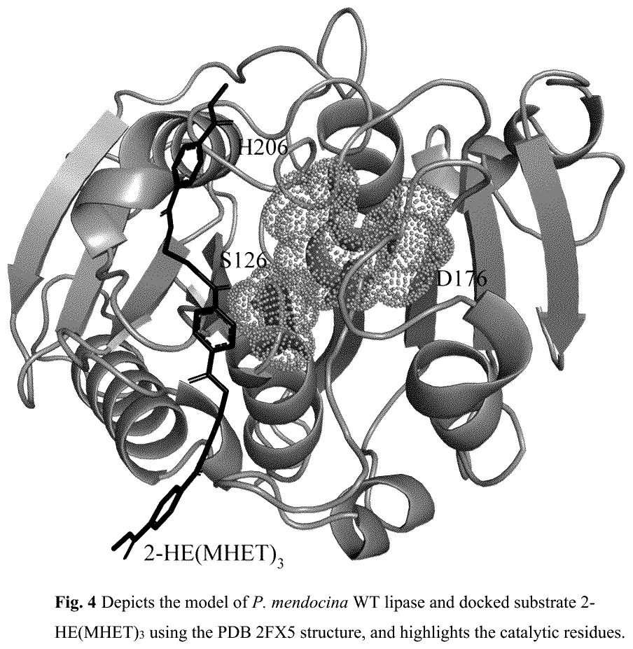

The three-dimensional structure of wild-type P. mendocina lipase (SEQ ID NO:2) determined by X-ray crystallographic method to 1.8 Å resolution and deposited by Bott et al on 2006 Mar. 2 as PDB 2PX5 was used for building a model of the putative substrate 2-HE(MHET)₃ docked in P. mendocina WT lipase (SEQ ID NO:2) to locate residues in proximity to substrate. The model was built by using MOE software (Chemical Computing Group ULC, V2020.090). A 3D image of the model of P. mendocina WT lipase with docked 2-HE(MHET)₃ was generated using PyMOLsoftware (Schrödinger, Inc., Version 2.3) and was used to compare the spatial location of evaluated amino acid substitutions with the benefit they imparted to the P. mendocina lipase variants described in these studies. FIG. 4 shows the P. mendocina WT lipase backbone as a grey ribbon, with the putative substrate 2-HE(MHET)₃ shown using black sticks, and the side chains of the catalytic residues S126/D176/H206 are shown as grey dotted spheres.

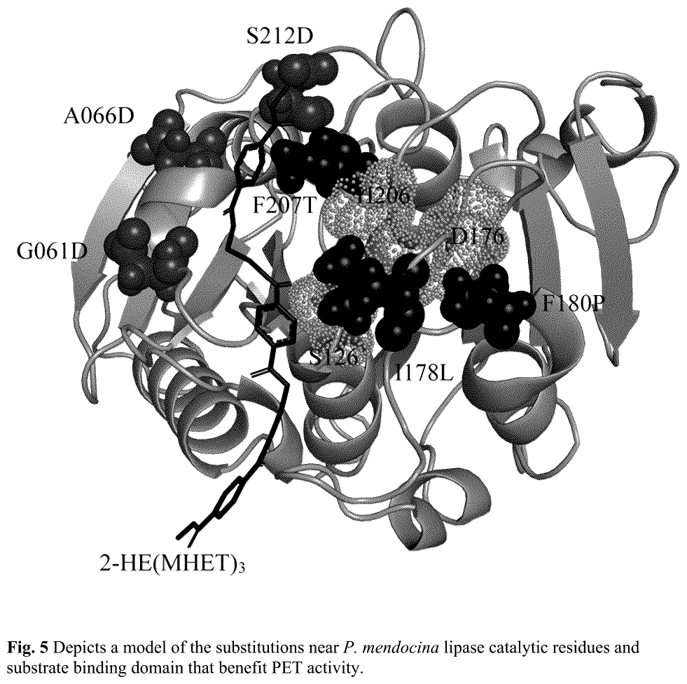

In FIG. 5, the representation of substitution sites for I178L, F180P, F207T, G061D, A066D, and S212D are generated on the P. mendocina WT lipase/2-HE(MHET)₃ model using MOE, where the lipase backbone is shown as grey ribbon and the putative 2-HE(MHET)₃ is shown as black stick. These amino acid substitution sites can be grouped to two clusters: the sites close to the catalytic residues and the sites near the substrate binding domain. I178L, F180P, and F207T substitution sites (shown as black spheres) are close to the S126/D176/H206 catalytic residues (shown as grey dots). We observed the distances between the Ca of I178L and three catalytic residues are 6.5A (D176), 6.6A (H206), and 8.3A (S126), respectively. In addition, the sidechain of I178L is close to these catalytic residues, ranging from 3.5 Å to 4.0 Å, with direct hydrophobic interaction between sidechains of I178L and H206. These close proximities to catalytic residues could explain the positive impact in the PET activity of the enzyme. These two substitutions, F180P and F207T also have major contribution to the improvement of PET activity and the overall performance of P. mendocina lipase according to the biochemical data obtained from the individual mutation variants shown in Example 2. In addition, the G061D, A066D, and S212D mutation sites shown as dark grey spheres are near the substrate binding domain and closed to the putative substrate 2-HE(MHET)₃. These three sites do not contact the substrate directly but may change the affinity to the substrate or the binding of the reaction product after hydrolysis, due to the additional negative charge contributed by the Asp residues.

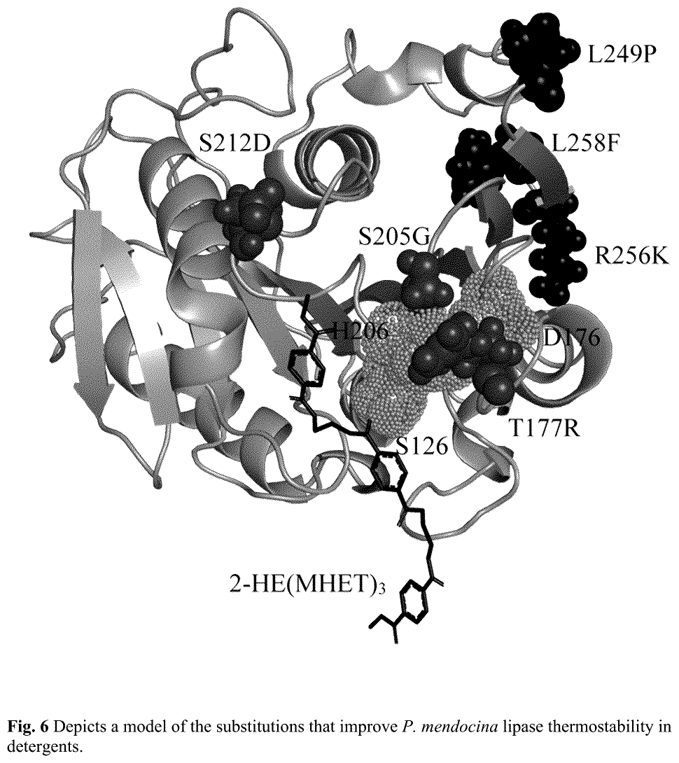

FIG. 6 shows the representation of substitution sites L249P, R256K, L258F, T177R, S205G, and S212D which are located at different regions of P. mendocina lipase. These substitutions make significant contributions on the improvement of the thermostability of P. mendocina lipase variants in detergents. It can be seen that residues L249P, R256K, and L258F are near the C-terminus, while residues T177R and S205G are near the catalytic residues, and residue S212D is near the substrate binding domain. The P. mendocina lipase backbone is shown as grey ribbon, and the modeled 2-HE(MHET)₃ substrate is shown as black sticks. The relative location of L249P, R256K, L258F, T177R, S205G, and S212D substitutions are shown as black and dark grey spheres, respectively. The substitution of L249P, R256K, and L258F changed the hydrophobicity and the charge of these residues could explain the improvement of the engineered lipases thermostability in detergents. In addition, T177R and S205G are close to the catalytic residues D176 and H206, while S212D is near the substrate binding domain of P. mendocina lipase. These substitutions also contribute to the P. mendocina lipase thermostability in detergents.

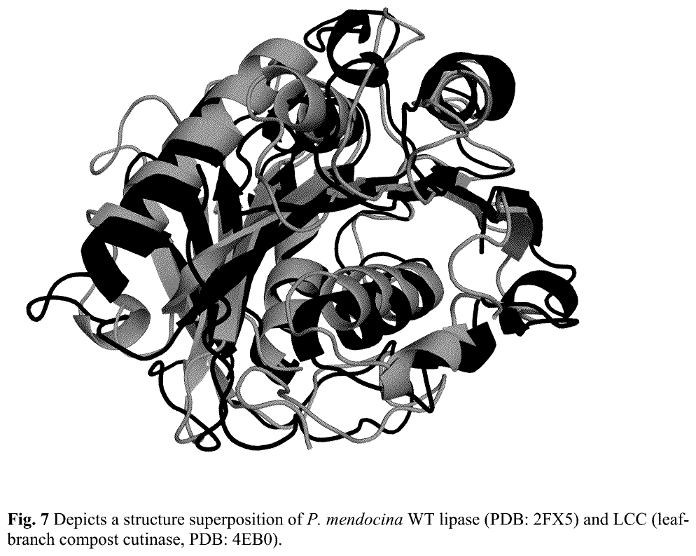

Our data indicate that the mutation sites near the catalytic residues and substrate binding domain can improve the performance significantly, while the substitution L249P, R256K, and L258F near the C-terminus can affect the thermostability of the molecule. The C-terminus of P. mendocina lipase is a flexible tail that plays an important role to stabilize the structural folding of the enzyme. The substitutions with the change of hydrophobicity and the charge of the residues may strengthen the interaction between the flexible C-terminal tail and the adjacent residues, thus further stabilizing the overall conformation of the enzyme. This is also supported by the data from other lipases/cutinases with similar structure folding. For example, although the amino acid sequence identity between P. mendocina lipase and LCC is low (21.7%), they share a common fold in 3D structure with an overall RMSD of 3.791 Angstrom. As shown in FIG. 7, structure superposition of P. mendocina lipase (PDB: 2FX5) and LCC (leaf-branch compost cutinase, PDB: 4EB0) was generated by PyMOL, where P. mendocina lipase is shown as black ribbon and LCC is shown as grey ribbon. In the U.S. Pat. No. 10,584,320 B2 (published on Mar. 10, 2020), the introduction of an additional disulfide bond near the C-terminus (D203C and S248C of LCC) was described and shown to improve the Tm of LCC molecule up to 9.50 Celsius (from 84.7 to 94.20 Celsius). The formation of a disulfide bond between adjacent domains is a common technique to strengthen the inter-chain interaction. The positive impact imparted by the substitutions of L249P, R256K, and L258F we introduced in the C-terminus of the P. mendocina lipase are in line with the results observed with disulfide bridges introduced into LCC.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

What is claimed is:

1. A variant lipolytic enzyme comprising an amino acid sequence having at least 70% identity to the full length amino acid sequence of SEQ ID NO: 2, comprising one or more substitutions at positions selected from the group consisting of 14, 70, 117, 161, 175, 212, 226, 236, 239, 252, 254, 256, and 258, wherein the positions are numbered by reference to the amino acid sequence of SEQ ID NO: 2, and wherein the variant has esterase activity.

2. The variant lipolytic enzyme of claim 1, wherein the variant comprises at least one additional mutation at a position selected from the group consisting of 40, 59, 61, 64, 66, 177, 178, 180, 182, 190, 205, 207, 210, and 249.

3. The variant lipolytic enzyme of claims 1 and 2, wherein the variant comprises one or more substitutions selected from the group consisting of V014S, R040A/T, G059Y, G061D, T064V, A066D, S07E, Q161H, G175A, R190L, S212D, F226L, A236P, Y239L, L249P, S252L, E254Q, R256K, and L258F, wherein the positions are numbered by reference to the amino acid sequence of SEQ ID NO: 2.

4. The variant lipolytic enzyme of any of the preceding claims, wherein the variant comprises an amino acid sequence having at least 75%, 80%, 85%, 90%, 91%, 92%, 93%, 94%, 95%, 96%, 97%, 98%, or 99% identity to the full length amino acid sequence of SEQ ID NO: 2.

5. A variant lipolytic enzyme comprising an amino acid sequence having at least 70% identity to the full length amino acid sequence of SEQ ID NO: 2, wherein the lipase variant comprises one, two, three, four, or more substitutions selected from the group consisting of V014S, R040A/T, G059Y, G061D, T064V, A066D, S07E, T117L, Q161H, G175A, T177N/R, I178L, F180P, Y182A/L, R190L, S205G, F207T, V210I, S212D, F226L, A236P, Y239L, L249P, S252L, E254Q, R256K, and L258F, wherein the positions are numbered by reference to the amino acid sequence of SEQ ID NO: 2.

6. The variant lipolytic enzyme of claim 5, wherein the variant has esterase activity.

7. The variant lipolytic enzyme of claim 5 or 6, wherein the variant comprises an amino acid sequence having at least 75%, 80%, 85%, 90%, 91%, 92%, 93%, 94%, 95%, 96%, 97%, 98%, or 99% identity to the full length amino acid sequence of SEQ ID NO: 2.

8. The variant lipolytic enzyme of any of the preceding claims, wherein the variant comprises the substitutions, or combination of substitutions, selected from the group consisting of R040A, G059Y, G061D, T064V, A066D, S07E, Q161H, F180P, Y182A/L, R190L, S205G, S212D, F226L, A236P, Y239I, L249P, S252I, E254Q, R256K, L258F, G061D-Y182A, G061D-S205G, F180P-Y182A, F180P-S205G, Y182A-S205G, V014S-R040A, V014S-R256K, R040A-E254Q, R040A-S205G, G059Y-A236P, G059Y-E254Q, R040A-F180P, G061D-F226L, G061D-R256K, T064V-F226L, A066D-F226L, A066D-L249P, S070E-L258F, T117L-S205G, T117L-L249P, Y182A-L249P, Q161H-Y239I, G175A-A236P, F180P-S205G, F180P-S252I, Y182A-S212D, Y182A-L258F, R190L-S205G, R190L-L249P, S205G-F207T, S205G-F226L, F207T-R256K, S212D-L249P, S212D-E254Q, F226L-R256K, Y239I-L249P, V014S-R040A-F180P, V014S-S205G-R256K, R040A-F226L-E254Q, G059Y-T117L-A236P, G059Y-A236P-E254Q, G059Y-L249P-L258F, G059Y-G061D-F226L, G061D-R256K-L258F, T064V-F226L-S252I, A066D-F226L-R256K, A066D-Y239I-L249P, S070E-S212D-L258F, T117L-S205G-F226L, T117L-F226L-A236P, T117L-S205G-S252I, Q161H-Y182A-A236P, Q161H-Y239I-L249P, G059Y-G175A-F180P, G175A-A236P-L258F, T064V-T177R-F180P, F180P-S205G-Y239I, R040A-F180P-S252I, T117L-Y182A-S212D, Y182A-F226L-L258F, T117L-R190L-L249P, S205G-F207T-A236P, S205G-F226L-L249P, G059Y-F207T-R256K, T064V-S212D-L249P, R040A-S212D-E254Q, F226L-S252I-R256K, Y239I-L249P-R256K, T177R-F180P-S205G, R040A-F180P-S205G, T064V-F180P-S205G, F180P-S205G-L249P, F180P-R190L-S205G, G061D-Y182A-S205G, F180P-Y182A-S205G, G061D-F180P-Y182A-S205G, and G061D-T117L-I178L-F180P-Y182A-R190L-S205G-F207T-S212D-F226L-Y239I-L249P-S252I-L258F, wherein the positions are numbered by reference to the amino acid sequence of SEQ ID NO: 2.

9. The variant lipolytic enzyme of any of the preceding claims, wherein the variant comprises the substitutions, or combination of substitutions, selected from the group consisting of R40T-T177N-F180P-S205G, V025T-R40T-T177N-F180P-S205G, R40T-T064V-T177N-F180P-S205G, R40T-T177N-F180P-Y182L-S205G, R40T-T177N-F180P-R190L-S205G, R40T-T177N-F180P-S205G-L249P, R40T-T064V-T177N-F180P-Y182L-S205G, R40T-T064V-T177N-F180P-R190L-S205G, R40T-T064V-T177N-F180P-S205G, R40T-T177N-F180P-Y182L-R190L-S205G, R40T-T177N-F180P-Y182L-S205G-L249P, R40T-T177N-F180P-R190L-S205G-L249P, R040T-T064V-T177N-F180P-Y182L-R190L-S205G-L249P, V025T-R40T-T064V-T177N-F180P-Y182L-R190L-S205G-L249P, R040T-T064V-T177N-F180P-Y182L-R190L-S205G-L249P, R040T-T064V-T177N-F180P-Y182A-R190L-S205G-L249P, V25T-R040T-T064V-T177N-F180P-Y182L-R190L-S205G-L249P, R040T-T064V-T177N-F180P-Y182L-R190L-S205G-L249P, R040T-T064V-T117L-T177N-I178L-F180P-Y182A-R190L-S205G-F207T-S212D-F226L-Y239I-L249P-S252I-L258F, R040A-T064V-T 117L-T177N-I178L-F180P-Y182A-R190L-S205G-F207T-S212D-F226L-Y239I-L249P-S252I-L258F, R040T-T064V-T117L-T177R-I178L-F180P-Y182A-R190L-S205G-F207T-S212D-F226L-Y239I-L249P-S252I-L258F, wherein the positions are numbered by reference to the amino acid sequence of SEQ ID NO: 2.

10. A variant lipolytic enzyme, or an active fragment thereof, comprising:
(a) an amino acid sequence having at least 70% identity to the full-length amino acid sequence of SEQ ID NO: 2,
(b) two or more substitutions at positions selected from
i) a substitution at position 61 and at least one additional substitution from a position selected from: 14, 40, 59, 64, 66, 70, 161, 175, 180, 182, 190, 205, 212, 226, 236, 239, 249, 252, 254, 256, and 258,
ii) a substitution at position 180 and at least one additional substitution at a position selected from 14, 40, 59, 61, 64, 66, 70, 161, 175, 182, 190, 205, 212, 226, 236, 239, 249, 252, 254, 256, and 258;
iii) a substitution at position 182 and at least one additional substitution at a position selected from 14, 40, 59, 61, 64, 66, 70, 161, 175, 180, 190, 205, 212, 226, 236, 239, 249, 252, 254, 256, and 258; or
iv) a substitution at position 205 and at least one additional substitution at a position selected from 40, 59, 61, 64, 66, 70, 161, 180, 182, 190, 212, 226, 236, 239, 249, 252, 254, 256, and 258;
wherein the positions are numbered by reference to the amino acid sequence of SEQ ID NO: 2, and wherein the variant lipolytic enzyme has esterase activity.

11. A variant lipolytic enzyme, or an active fragment thereof, comprising:
(a) an amino acid sequence having at least 70% identity to the full-length amino acid sequence of SEQ ID NO: 2,
(b) a combination of mutations selected from the group consisting of
(i) a combination of mutations G061D-Y182A/L and at least one additional mutation selected from the group consisting of V014S, R040A/T, G059Y, T064V, A066D, S07E, T117L, Q161H, G175A, T177N/R, I178L, F180P, R190L, S205G, F207T, V210I, S212D, F226L, A236P, Y239I, L249P, S252I, E254Q, R256K, and L258F,
(ii) a combination of mutations G061D-S205G and at least one additional mutation selected from the group consisting of V014S, R040A/T, G059Y, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
(iii) a combination of mutations F180P-Y182A/L and at least one additional mutation selected from the group consisting of V014S, R040A/T, G059Y, G061D, T064V, A066D, S07E, T117L, Q161H, G175A, T177N/R, I178L, R190L, S205G, F207T, V210I, S212D, F226L, A236P, Y239I, L249P, S252I, E254Q, R256K, and L258F,
(iv) a combination of mutations F180P-S205G and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F,
(v) a combination of mutations Y182A/L-S205G and at least one additional mutation selected from the group consisting of V014S, R040A/T, G059Y, G061D, T064V, A066D, S07E, T117L, Q161H, G175A, T177N/R, I178L, F180P, R190L, F207T, V210I, S212D, F226L, A236P, Y239I, L249P, S252I, E254Q, R256K, and L258F,
(vi) a combination of mutations V014S-R040A and at least one additional mutation selected from the group consisting of G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, R256K, and L258F,
(vii) a combination of mutations V014S-R256K and at least one additional mutation selected from the group consisting of R040A/T, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
(viii) a combination of mutations R040A-E254Q and at least one additional mutation selected from the group consisting of V014S, G059Y, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
(ix) a combination of mutations R040A-S205G and at least one additional mutation selected from the group consisting of V014S, G059Y, G061D, T064V, A066D, S07E, T117L, Q161H, G175A, T177N/R, I178L, F180P, Y182A/L, R190L, F207T, V210I, S212D, F226L, A236P, Y239I, L249P, S252I, E254Q, R256K, and L258F,
(x) a combination of mutations G059Y-A236P and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, Y239I, L249P, S252I, E254Q, R256K, and L258F,
(xi) a combination of mutations G059Y-E254Q and at least one additional mutation selected from the group consisting of V014S, R040A/T, G061D, T064V, A066D, S07E, T117L, Q161H, G175A, T177N/R, I178L, F180P, Y182A/L, R190L, S205G, F207T, V210I, S212D, F226L, A236P, Y239I, L249P, S252I, R256K, and L258F,
(xii) a combination of mutations R040A-F180P and at least one additional mutation selected from the group consisting of V014S, G059Y, G061D, T064V, A066D, S07E, T117L, Q161H, G175A, T177N/R, I178L, Y182A/L, R190L, S205G, F207T, V210I, S212D, F226L, A236P, Y239I, L249P, S252I, E254Q, R256K, and L258F,
(xiii) a combination of mutations G061D-F226L and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
(xiv) a combination of mutations G061D-R256K and at least one additional mutation selected from the group consisting of V014S, R040A/T, G059Y, T064V, A066D, S07E, T117L, Q161H, G175A, T177N/R, I178L, F180P, Y182A/L, R190L, S205G, F207T, V210I, S212D, F226L, A236P, Y239I, L249P, S252I, E254Q, and L258F,
(xv) a combination of mutations T064V-F226L and at least one additional mutation selected from the group consisting of V014S, R040A/T, G059Y, G061D, A066D, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
(xvi) a combination of mutations A066D-F226L and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, A236P, Y239I, L249P, S252I, E254Q, R256K, and L258F,
(xvii) a combination of mutations A066D-L249P and at least one additional mutation selected from the group consisting of V014S, R040A/T, G059Y, G061D, T064V, S07E, T117L, Q161H, G175A, T177N/R, I178L, F180P, Y182A/L, R190L, S205G, F207T, V210I, S212D, F226L, A236P, Y239I, S252I, E254Q, R256K, and L258F,
(xviii) a combination of mutations S070E-L258F and at least one additional mutation selected from the group consisting of V014S, R040A/T, G059Y, G061D, T064V, A066D, T117L, Q161H, G175A, T177N/R, I178L, F180P, Y182A/L, R190L, S205G, F207T, V210I, S212D, F226L, A236P, Y239I, L249P, S252I, E254Q, and R256K,
(xix) a combination of mutations T117L-S205G and at least one additional mutation selected from the group consisting of V014S, R040A/T, G059Y, G061D, T064V, A066D, S07E, Q161H, G175A, T177N/R, I178L, F180P, Y182A/L, R190L, F207T, V210I, S212D, F226L, A236P, Y239I, L249P, S252I, E254Q, R256K, and L258F,
(xx) a combination of mutations T117L-L249P and at least one additional mutation selected from the group consisting of V014S, R040A/T, G059Y, G061D, T064V, A066D, S07E, Q161H, G175A, T177N/R, I178L, F180P, Y182A/L, R190L, S205G, F207T, V210I, S212D, F226L, A236P, Y239I, S252I, E254Q, R256K, and L258F,
(xxi) a combination of mutations Y182A/L-L249P and at least one additional mutation selected from the group consisting of V014S, R040A/T, G059Y, G061D, T064V, A066D, S07E, T117L, Q161H, G175A, T177N/R, I178L, F180P, R190L, S205G, F207T, V210I, S212D, F226L, A236P, Y239I, S252I, E254Q, R256K, and L258F,
(xxii) a combination of mutations Q161H-Y239I and at least one additional mutation selected from the group consisting of V014S, R040A/T, G059Y, G061D, T064V, A066D, S07E, T117L, G175A, T177N/R, I178L, F180P, Y182A/L, R190L, S205G, F207T, V210I, S212D, F226L, A236P, L249P, S252I, E254Q, R256K, and L258F, and
(xxiii) a combination of mutations G175A-A236P and at least one additional mutation selected from the group consisting of V014S, R040A/T, G059Y, G061D, T064V, A066D, S07E, T117L, Q161H, T177N/R, I178L, F180P, Y182A/L, R190L, S205G, F207T, V210I, S212D, F226L, Y239I, L249P, S252I, E254Q, R256K, and L258F, wherein the positions are numbered by reference to the amino acid sequence of SEQ ID NO: 2, and wherein the variant lipolytic enzyme has esterase activity.

12. A variant lipolytic enzyme comprising:
(a) an amino acid sequence having at least 70% identity to the full length amino acid sequence of SEQ ID NO: 2,
(b) a combination of mutations selected from the group consisting of
(i) T177N/R-F180P-S205G and at least one additional mutation selected from the group consisting of V014S, R040A/T, G059Y, G061D, T064V, A066D, S07E, T117L, Q161H, G175A, I178L, Y182A/L, R190L, F207T, V210I, S212D, F226L, A236P, Y239I, L249P, S252I, E254Q, R256K, and L258F;
(ii) R040A/T-F180P-S205G and at least one additional mutation selected from the group consisting of V014S, G059Y, G061D, T064V, A066D, S07E, T117L, Q161H, G175A, T177N/R I178L, Y182A/L, R190L, F207T, V210L, S212D, F226L, A236P, Y239I, L249P, S252I, E254Q, R256K, and L258F;
(iii) G059Y-F180P-S205G and at least one additional mutation selected from the group consisting of V014S, R040A/T, G061D, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
(iv) G061D-F180P-S205G and at least one additional mutation selected from the group consisting of V014S, R040A/T, G059Y, T064V, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
(v) T064V-F180P-S205G and at least one additional mutation selected from the group consisting of V014S, R040A/T, G059Y, G061D, A066D, S07E, T117L, Q161H, G175A, T177N/R, I178L, Y182A/L, R190L, F207T, V210I, S212D, F226L, A236P, Y239I, L249P, S252I, E254Q, R256K, and L258F;
(vi) A066D-F180P-S205G and at least one additional mutation selected from the group consisting of V014S, R040A/T, G059Y, G061D, T064V, S07E, T117L, Q161H, G175A, T177N/R, I178L, Y182A/L, R190L, F207T, V210I, S212D, F226L, A236P, Y239I, L249P, S252I, E254Q, R256K, and L258F;
(vii) S070E-F180P-S205G and at least one additional mutation selected from the group consisting of V014S, R040A/T, G059Y, G061D, T064V, A066D, T117L, Q161H, G175A, T177N/R, I178L, Y182A/L, R190L, F207T, V210I, S212D, F226L, A236P, Y239I, L249P, S252I, E254Q, R256K, and L258F;
(viii) T117L-F180P-S205G and at least one additional mutation selected from the group consisting of V014S, R040A/T, G059Y, G061D, T064V, A066D, S07E, Q161H, G175A, T177N/R, I178L, Y182A/L, R190L, F207T, V210I, S212D, F226L, A236P, Y239I, L249P, S252I, E254Q, R256K, and L258F;
(ix) Q161H-F180P-S205G and at least one additional mutation selected from the group consisting of V014S, R040A/T, G059Y, G061D, T064V, A066D, S07E, T117L, G175A, T177N/R, I178L, Y182A/L, R190L, F207T, V210I, S212D, F226L, A236P, Y239I, L249P, S252I, E254Q, R256K, and L258F;
(x) I178L-F180P-S205G and at least one additional mutation selected from the group consisting of V014S, R040A/T, G059Y, G061D, T064V, A066D, S07E, T117L, Q161H, G175A, T177N/R, Y182A/L, R190L, F207T, V210I, S212D, F226L, A236P, Y239I, L249P, S252I, E254Q, R256K, and L258F;
(xi) F180P-Y182A/L-S205G and at least one additional mutation selected from the group consisting of V014S, R040A/T, G059Y, G061D, T064V, A066D, S07E, T117L, Q161H, G175A, T177N/R, I178L, R190L, F207T, V210I, S212D, F226L, A236P, Y239I, L249P, S252I, E254Q, R256K, and L258F;
(xii) F180P-R190L-S205G and at least one additional mutation selected from the group consisting of V014S, R040A/T, G059Y, G061D, T064V, A066D, S07E, T117L, Q161H, G175A, T177N/R, I178L, Y182A/L, F207T, V210I, S212D, F226L, A236P, Y239I, L249P, S252I, E254Q, R256K, and L258F;
(xiii) F180P-S205G-F207T and at least one additional mutation selected from the group consisting of V014S, R040A/T, G059Y, G061D, T064V, A066D, S07E, T117L, Q161H, G175A, T177N/R, I178L, Y182A/L, R190L, V210I, S212D, F226L, A236P, Y239I, L249P, S252I, E254Q, R256K, and L258F;
(xiv) F180P-S205G-V210I and at least one additional mutation selected from the group consisting of V014S, R040A/T, G059Y, G061D, T064V, A066D, S07E, T117L, Q161H, G175A, T177N/R, I178L, Y182A/L, R190L, F207T, S212D, F226L, A236P, Y239I, L249P, S252I, E254Q, R256K, and L258F;
(xv) F180P-S205G-S212D and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, F226L, A236P, Y239I, L249P, S252I, E254Q, R256K, and L258F;
(xvi) F180P-S205G-F226L and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, A236P, Y239I, L249P, S252I, E254Q, R256K, and L258F;
(xvii) F180P-S205G-A236P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, Y239I, L249P, S252I, E254Q, R256K, and L258F;
(xviii) F180P-S205G-Y239I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, L249P, S252I, E254Q, R256K, and L258F;
(xix) F180P-S205G-L249P and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, S252I, E254Q, R256K, and L258F;
(xx) F180P-S205G-S252I and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, E254Q, R256K, and L258F;
(xxi) F180P-S205G-E254Q and at least one additional mutation selected from the group consisting of V014S, R040A/T, G059Y, G061D, T064V, A066D, S07E, T117L, Q161H, G175A, T177N/R, I178L, Y182A/L, R190L, F207T, V210I, S212D, F226L, A236P, Y239I, L249P, S252I, R256K, and L258F;
(xxii) F180P-S205G-R256K and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and L258F; and
(xxiii) F180P-S205G-L258F and at least one additional mutation selected from the group consisting of V014S, R040A/T, G059Y, G061D, T064V, A066D, S07E, T117L, Q161H, G175A, T177N/R, I178L, Y182A/L, R190L, F207T, V210L, S212D, F226L, A236P, Y239I, L249P, S252I, E254Q, and R256K wherein the positions are numbered by reference to the amino acid sequence of SEQ ID NO: 2, and wherein the variant lipolytic enzyme has esterase activity.

13. The variant lipolytic enzyme of claim 11 or 12, wherein the variant comprises an amino acid sequence having at least 75%, 80%, 85%, 90%, 91%, 92%, 93%, 94%, 95%, 96%, 97%, 98%, or 99% identity to the full length amino acid sequence of SEQ ID NO: 2.

14. The variant lipolytic enzyme of and of claims 11-13, wherein the variant comprises a combination of mutations selected from the group consisting of R040T-T064V-T177N-F180P-Y182L-R190L-S205G-L249P, and R040T-T064V-T117L-T177N-I178L-F180P-Y182A-R190L-S205G-F207T-S212D-F226L-Y239I-L249P-S252I-L258F, wherein the positions are numbered by reference to the amino acid sequence of SEQ ID NO: 2.

15. The variant lipolytic enzyme of any of the preceding claims, wherein the variant has one or more improved properties when compared to a parent or reference lipolytic enzyme, wherein the improved property is selected from improved stability, improved hydrolytic activity on a polyester, or combinations thereof.

16. The variant lipolytic enzyme of any of claim 15, wherein the improved property is:
(i) improved stability, wherein said variant has a residual activity at least 5% when measured in accordance with the stability assay of Example 2 and/or
(ii) improved hydrolytic activity on a polyester, wherein said variant has a PI≥1.1 compared to the lipase having the amino acid sequence of SEQ ID NO: 2 when measured in accordance with the PET assay of Example 2.

17. A polynucleotide comprising a nucleic acid sequence encoding a variant lipolytic enzyme of any one of claims 1-16.

18. The polynucleotide of claim 17, wherein the nucleic acid sequence is operably linked to a promoter.

19. An expression vector or cassette comprising the polynucleotide of claim 17 or 18.

20. A recombinant host cell comprising the expression vector or cassette of claim 19.

21. An enzyme composition comprising a variant lipolytic enzyme of any one of claims 1-16.

22. A cleaning composition or detergent composition comprising a variant lipolytic enzyme of any one of claims 1-16 and at least one adjunct selected from the group consisting of surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

23. The cleaning or detergent composition of claim 22, wherein the composition further comprises at least one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, lysozymes, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

24. A fabric treatment composition comprising a variant lipolytic enzyme of any one of claims 1-16 and at least one adjunct selected from the group consisting of surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

25. The fabric treatment composition of claim 24, wherein the composition further comprises at least one additional enzyme selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, lysozymes, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

26. A method for treating a fabric or textile comprising, (i) contacting a fabric or textile with a variant lipolytic enzyme of any one of claims 1-16 or a composition comprising said variant lipolytic enzyme, and (ii) optionally, rinsing the fabric or textile.

27. The method of claim 26, wherein the fabric or textile comprises at least one polyester.

28. The method of any of claims 26 and 27, wherein the contacting step comprises a variant lipolytic enzyme in an amount selected from the group consisting of a) 0.002 to 10,000 mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or b) in the amount of at least 0.01 ppm active enzyme.

29. A method for degrading a polyester or a polyester containing material comprising
i) contacting the polyester containing material with a variant lipolytic enzyme according to any one of claims 1-16 or a composition of claims 22-25, and, optionally,
ii) rinsing said polyester containing material.

30. A method for the enzymatic depolymerization of a polyester or a polyester containing material comprising,
i) contacting the polyester or polyester containing material with a variant lipolytic enzyme according to any one of claims 1-16 or a composition of claims 22-25, and, optionally,
ii) recovering monomers and/or oligomers of the polyester.

31. The method of claim 29 or 30, wherein the polyester is selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.

32. The lipolytic enzyme of any of claims 1-16, wherein the variant has lypolytic activity on a polyester selected from the group consisting of polyethylene terephthalate (PET), polytrimethylene terephthalate (PTT), polybutylene terephthalate (PBT), polyethylene isosorbide terephthalate (PEIT), polylactic acid (PLA), polyhydroxy alkanoate (PHA), polybutylene succinate (PBS), polybutylene succinate adipate (PBSA), polybutylene adipate terephthalate (PBAT), polyethylene furanoate (PEF), polycaprolactone (PCL), polyethylene naphthalate (PEN), polyester polyurethane, poly(ethylene adipate) (PEA), and combinations thereof.
